(19) United States
(12) Patent Application Publication
(10) Pub. No.: US 2018/0105863 A1
(43) Pub. Date: Apr. 19, 2018

(54) NEURAL STEM CELL THERAPY FOR OBESITY AND DIABETES

(71) Applicant: ALBERT EINSTEIN COLLEGE OF MEDICINE, INC., Bronx, NY (US)

(72) Inventor: Dongsheng Cai, Larchmont, NY (US)

(73) Assignee: ALBERT EINSTEIN COLLEGE OF MEDICINE, INC., Bronx, NY (US)

(21) Appl. No.: 15/783,100

(22) Filed: Oct. 13, 2017

Related U.S. Application Data
(62) Division of application No. 14/375,582, filed on Jul. 30, 2014, now abandoned.
(60) Provisional application No. 61/593,557, filed on Feb. 1, 2012.

(51) Int. Cl. C12Q 1/48; C12N 5/0797; A61K 35/30; G01N 33/50; A61K 35/12

(52) U.S. Cl. CPC C12Q 1/485; C12N 5/0623; G01N 2333/91215; G01N 33/5088; A61K 35/12; A61K 35/30

(57) ABSTRACT

Methods are provided of treating obesity or an obesity comorbidity in a mammalian subject comprising administering to the subject an amount of an agent effective to treat obesity or the obesity comorbidity, which agent inhibits (i) IκB kinase (IKKβ) activation of nuclear factor kappa-light-chain-enhancer of activated B cells (NF-κB) or (ii) Notch signaling, in a manner so as to permit the agent to enter the hypothalamus of the subject. Assays are also provided for identifying candidate agents for treating obesity’.

### CROSS-REFERENCE TO RELATED APPLICATIONS

This application claims benefit of U.S. Provisional Application No. 61/593,557, filed Feb. 1, 2012, the contents of which are hereby incorporated by reference.

### STATEMENT OF GOVERNMENT SUPPORT

This invention was made with government support under grant numbers R01 DK078750 and R01 AG031774 awarded by the National Institutes of Health. The government has certain rights in the invention.

### BACKGROUND OF THE INVENTION

Throughout this application various publications are referred to in parentheses by author and year of publication. The disclosures of these publications, and all patents and patent application publications and books referred to herein, are hereby incorporated by reference in their entirety into the subject application to more fully describe the art to which the subject invention pertains.

The hypothalamus in the central nervous system (CNS) is a fundamental regulator of many life-supporting biological processes, such as growth, reproduction, stress response, sleep-awake cycle, fluid and salt balance, body temperature, feeding, body weight, and glucose metabolism. During recent years, devastating surges in the incidence of obesity, type 2 diabetes (T2D) and their complications have been actively stimulating the research to elucidate the neuronal subtypes and molecular pathways in the hypothalamic control of body weight and metabolic homeostasis (Niswender et al., 2004; Munzberg and Myers, Jr., 2005; Flier, 2006; Coll et al., 2007). These research endeavors have led to the important establishment of multiple hypothalamic molecular and cellular models which were grounded on the view that adult neurons are non-replenishable.

Only very recently has this belief begun to be challenged by the identification of adult neural stem cells (NSC) in a few brain regions, predominately in the sub-ventricular zone (SVZ) of the forebrain and the sub-granular zone (SGZ) of the hippocampal dentate gyrus (Reynolds and Weiss, 1992; Ray et al., 1993; Cameron and McKay, 1998; Johansson et al., 1999; Gage, 2000; Gross, 2000; Morrison, 2001; Temple, 2001; varez-Buylla and Lim, 2004; Emsley et al., 2005; Gould, 2007; Whitman and Greer, 2009). In light of the hypothalamus, a few recent studies have observed hypothalamic activities of neurogenesis in adult mice (Kokoeva et al., 2005; Kokoeva et al., 2007; Pierce and Xu, 2010) and rats (Pencea et al., 2001), supporting a concept that the postnatal hypothalamic development may contribute to the regulation of metabolic physiology (Bouret et al., 2004; Bouret et al., 2008). However, the predictable existence of adult hypothalamic NSC has hitherto not been studied for either physiological function or disease relevance, and in particular the question of whether NSC might have value for disease intervention and if so, how, has not been investigated.

The development of obesity and T2D under the obesogenic environment is etiologically associated with the onset of chronic inflammation in metabolic tissues (Ruan and Lodish, 2004; Hotamisligil, 2006; Shoelson and Goldfine, 2009; Cai, 2009). The pro-inflammatory molecular pathways that interrupt the functions and regulations of peripheral metabolic tissues have been identified to include nuclear factor κB (NF-κB) and its upstream signaling activator IκB kinase (IKKβ) (Yuan et al., 2001; Shoelson and Goldfine, 2009). More recently, IKKβ/NF-κB was revealed to mediate hypothalamic inflammation which promotes the development of energy imbalance, insulin resistance and related metabolic syndrome (Zhang et al., 2008; Purkayastha et al., 2011b). Notably, in addition to being an inflammation/immunity regulator, IKKβ/NF-κB can control cell growth, apoptosis and differentiation in dynamic and cell-specific manners (Hayden et al., 2006; Hoffmann and Baltimore, 2006; Li and Verma, 2002; Karin and Lin, 2002). In fact, NF-κB can be pro-survival or anti-survival depending on cell types and pathological context (Dutta et al., 2006; Vousden, 2009), but the profile pertaining to how IKKβ/NF-κB could affect NSC still represents a poorly appreciated subject. Despite that little available evidence has related NF-κB to the effects of cytokines/growth factors or stresses on the neurogenetic activity of the hippocampus (Rolls et al., 2007; Koo and Duman, 2008; Koo et al., 2010; is-Donini et al., 2008), the hypothalamus has not been examined in terms of whether IKKβ/NF-κB might employ a neurogenetic program to affect metabolic physiology.

The current invention identifies a novel pathway in the obesogenic cycle and provides therapies based thereon.

### SUMMARY OF THE INVENTION

A method is provided of treating obesity or an obesity comorbidity in a mammalian subject comprising administering to the subject an amount of an agent effective to treat obesity or the obesity comorbidity, which agent inhibits (i) IκB kinase β (IKKβ) activation of nuclear factor kappa-light-chain-enhancer of activated B cells (NF-κB) or (ii) Notch signaling, in a manner so as to permit the agent to enter the hypothalamus of the subject, so as to treat the obesity or the obesity comorbidity in the subject.

Also provided is a method of identifying an agent as a candidate treatment for obesity or an obesity comorbidity in a subject, the method comprising testing if the agent inhibits IKKβ/NF-κB activation by contacting the IKKβ and/or NF-κB with the agent, and determining if the agent is an inhibitor of IKKβ/NF-κB activation, wherein if the agent does not inhibit IKKβ/NF-κB activation it is not a candidate treatment, and wherein if the agent does inhibit IKKβ/NF-κB activation it is a candidate treatment.

Also provided is a method of identifying an agent as a candidate treatment for obesity or an obesity comorbidity in a subject, the method comprising testing whether the agent inhibits IKKβ/NF-κB in the hypothalamus of a non-human mammal, and determining if the agent is an inhibitor of IKKβ/NF-κB activation in the hypothalamus, wherein if the agent inhibits IKKβ/NF-κB in the hypothalamus of the non-human mammal it is a candidate treatment, and wherein if the agent does not inhibit IKKβ/NF-κB in the hypothalamus of the non-human mammal it is not a candidate treatment.

Also provided is a method of identifying an agent as a candidate treatment for obesity or an obesity comorbidity in a subject, the method comprising testing if the agent inhibits a Notch 1, Notch 2, Notch 3 or Notch 4 in the hypothalamus of a non-human mammal, and determining of the agent is an inhibitor of a Notch 1, Notch 2, Notch 3 or Notch 4 in the hypothalamus,

wherein if the agent inhibits Notch 1, Notch 2, Notch 3 or Notch 4 in the hypothalamus of the non-human mammal it is a candidate treatment, and wherein if the agent does not inhibit Notch 1, Notch 2, Notch 3 or Notch 4 in the hypothalamus of the non-human mammal it is not a candidate treatment.

A pharmaceutical composition is provided for treating obesity or an obesity comorbidity, comprising an inducible pluripotent cell comprising a heterologous nucleic acid or having a genetic sequence deleted therein or a neural stem cell comprising a heterologous nucleic acid or having a genetic sequence deleted therein and a pharmaceutically acceptable carrier, wherein the inducible pluripotent cell or neural stem cell comprises a a heterologous nucleic acid encoding a dominant-negative IκBα or comprises a dominant-negative IκBα transfected via means of a viral vector, or has a IKKβ genetic sequence deleted, or comprises a shRNA directed against a Notch 1, Notch 2, Notch 3 or Notch 4.

An inducible pluripotent cell is provided comprising a heterologous nucleic acid or having a genetic sequence deleted therein or a neural stem cell comprising a heterologous nucleic acid or having a genetic sequence deleted therein and a pharmaceutically acceptable carrier, wherein the inducible pluripotent cell or neural stem cell comprises a heterologous nucleic acid encoding a dominant-negative IκBα (DN IκBα) or comprises a dominant-negative IκBα transfected via means of a viral vector, or has a IKKβ genetic sequence deleted, or comprises a shRNA directed against a Notch 1, Notch 2, Notch 3 or Notch 4, for treating obesity or an obesity comorbidity.

Additional objects of the invention will be apparent from the description which follows.

### BRIEF DESCRIPTION OF THE DRAWINGS

FIG. 1A-1E. In vivo and in vitro characterization of adult hNSC. A. Brain sections across the hypothalamus and other brain regions were prepared from C57BL/6 mice (chow-fed males, 3 months old) for co-immunostaining of Sox2 (green) and NeuN (red). Nuclear staining (blue) by DAPI revealed all cells in the sections. Merged images show the co-distribution of Sox2 with DAPI (Sox2+DAPI) but not NeuN (Sox2+NeuN). 3V: third ventricle; ARC: arcuate nucleus; DG: dentate gyrus; LV: lateral ventricle. Scale bar=50 μm. B&C. Hypothalamic tissues were sampled from C57BL/6 mice (chow-fed males, 3 months old) for neurosphere culture as described in Methods section. Neurospheres were formed and passaged in growth medium containing bFGF and EGF. Neurospheres at various passages were attached to slides for immunostaining of Sox2 (B) and co-staining of nestin and Blbp (C). Images were merged with DAPI staining to reveal nuclear distribution of Sox2 and cytoplasmic distribution of nestin and Blbp. Bar=50 μm. Data represent similar profiles at Passages 1-10. D. The hypothalamus and various other brain components were sampled from C57BL/6 mice (chow-fed males, 3 months old) for neurosphere assay. Data shows the total numbers of primary neurospheres (without passaging) normalized by the mass (mg) of neurospheres-derived brain tissues. Hy: hypothalamus; Co: cortex; Po: pons; Th: thalamus; Ce: cerebellum; DG: dentate gyrus. **P<0.01, ***P<0.001, n=5 mice/group; error bars reflect means±SEM. E. Neurospheres were derived from the hypothalamus of mice (chow-fed males, 3 months old). Dissociated neurospheric cells at the same passage were subjected to 7-day neural differentiation, and examined for immunostaining (green) of neuronal marker Tuj1, astrocyte marker GFAP, and oligodendrocyte marker 04. Nuclear staining (blue) by DAPI revealed the entire populations of cells. Bar=50 μm. Data represented similar observations in cells at Passages 1-10.

FIG. 2A-2O. Adult hNSC-derived neurogenesis and metabolic function in mice. A-D. C57BL/6 mice (chow-fed males, 4 months old) received Brdu injection, and at various time post Brdu injection, hypothalamus sections were generated for staining Brdu-labeled cells. A&B: Co-immunostaining of Brdu (red) and NeuN (green) (A) or POMC (green) (B) at Day 10 vs. Day 30 post Brdu injection. Nuclear staining (blue) by DAPI revealed all cells in the sections and the nuclear localization of Brdu. C&D: Numbers of Brdu-labeled NeuN-positive cells (Brdu+NeuN+) (C) and POMC-positive (Brdu+POMC+) (D) in the arcuate nucleus (ARC) at indicated days post injection. Cell numbers were counted based on serial ARC sections. E-H. ROSA-lox-STOP-lox-YFP mice (chow-fed males, 3 month old) were bilaterally injected in the mediobasal hypothalamus with lentiviruses which directed Cre expression under the control of Sox2 promoter. Following the indicated periods post injection, hypothalamus sections were generated for tracking neural differentiation of GFP-labeled cells. E&F: Co-imaging of YFP (green) (E&F) with immunostaining (red) of Sox2 (E) and NeuN (F) at indicated days post viral injection. Nuclear staining (blue) by DAPI revealed all cells in the sections. G&H: Numbers of YFP-labeled NeuN-positive cells (Brdu+NeuN+) (G) and YFP-labeled POMC-positive cells (Brdu+POMC+) (H) in the arcuate nucleus (ARC). Cell numbers were counted based on serial ARC sections. I-O. C57BL/6 mice (chow-fed males, 4 month old) were bilaterally injected in the mediobasal hypothalamus with Psox2-Hsv1-TK lentiviruses vs. control lentiviruses (data not shown) and then maintained on GCV-containing drinking water and under chow feeding during the experiment. I: Sox2 immunostaining in the mediobasal hypothalamus of mice at ˜12 weeks post lentivirus injection. J-O: Daily food intake (J), food intake normalized by lean mass (K), O₂ consumption normalized by lean mass (L), body weight (M), area under curve (AUC) of GTT (N), and fasting blood insulin levels (0) of mice. Data were obtained at Week 12˜13 (J-L, N, O) or Week 0 vs. 12 (M) post viral injection. ARC: arcuate nucleus; VMH: ventral medial hypothalamic nucleus; 3V: third ventricle; H-TK: mice injected with Psox2-Hsv1-TK lentivirus; Con: mice injected with control lentivirus. *P<0.05, **P<0.01, ***P<0.001, n=4-6 per group (C, D, G, H) and n=6-8 per group (J-O). Error bars reflect means±SEM. au: arbitrary unit. Scale bar=25 μm (A, B, E, F, I).

FIG. 3A-3N. Chronic HFD feeding impairs hNSC and related neurogenesis. A-F. Male C57BL/6 mice were maintained under chow vs. HFD feeding for 4 months (A&B, D-F) or 8 months (C), and analyzed for Sox2 immunostaining (A&B), neuronal staining (C) and Brdu labeling (D-F). A&B: Sox2-positive (Sox2+) cells in the mediobasal hypothalamic sections were immunostained (green) (A) and counted (B). Nuclear staining by DAPI (blue) revealed all cells in the sections (A). C: Numbers of neurons in the arcuate nucleus were counted via NeuN immunostaining (images not shown). D-F: Mediobasal hypothalamic sections of Brdu-injected mice were examined for Brdu-positive (Brdu+) cells (D&E) and further analyzed for the fraction of NeuN-positive (NeuN+) cells (F) via co-immunostaining (images not shown). Nuclear staining by DAPI (blue) revealed all cells in the sections (D). G-N. Hypothalamic neurospheres were obtained from male C57BL/6 mice that received chow vs. HFD feeding for 4 months since weaning. Neurospheric cells were cultured and analyzed for morphology (G-I), proliferation (J), and differentiation (K-N). G: Representative images of primary neurospheres derived from chow-fed vs. HFD-fed mice. H&I: Average numbers (per hypothalamus) and size of primary neurospheres (NS) from the hypothalamus of chow- vs. HFD-fed mice. J: Cell outputs from the same initial number (104 cells) of primary neurospheric cells over 5 generations of passaging. Chow and HFD indicate the primary neurospheres derived from chow- vs. HFD-fed mice. K-N: Neurospheric cells derived from chow- vs. HFD-fed mice were subjected to 7-day differentiation at the same passages, and immunostained (green) for neuronal marker Tuj1 (K) and astrocyte marker GFAP (M). Images were merged with DAPI nuclear staining (blue) to show all cells. Bar graphs: Percentage of Tuj1-positive (Tuj1+) cells (L) and GFAP-positive (GFAP+) cells (N). Data in K-N represent similar observations at Passages 5-10. *P<0.05, **P<0.01, ***P<0.001, n=4-6 mice per group (A-N) Error bars reflect means±SEM. Scale bar=50 μm (A, D, G, K, M). 3V: third ventricle; ARC: arcuate nucleus; VMH: ventral medial hypothalamic nucleus (A&D).

FIG. 4A-40. IKKβ/NF-κB mediates HFD to impair hNSC survival and differentiation. A-C. NSC were derived from the hypothalamus of C57BL/6 mice that received 4-month HFD vs. chow feeding (A), and normal mice-derived hNSC were transduced with stable expression of CAIKKβ, DNIκBα or control GFP (B&C). Data show Western blot analysis of these cell models at Passage 4-6 for IKKβ/NF-κB signaling proteins. Bar graphs: quantitation of Western blots. D-I. CAIKKβ-hNSC, DNIκBα-hNSC and control GFP-hNSC. FIG. 3E-G at a low passage (Passage 6) were subjected to Brdu labeling (D&E) and cell output assay (F), and neural differentiation (G-I). D&E: Same numbers of dissociated cells were cultured in growth medium and were pulse labeled with Brdu at Day 3. Cells in slides were stained for Brdu (red) (D) and counted for the percentage of Brdu-positive (Brdu+) cells (E). The entire populations of cells were visualized by GFP (green) and DAPI staining (blue). F: Same numbers of dissociated cells were cultured in growth medium and analyzed for cell outputs over 5 passages. G-I: Same numbers of dissociated cells were subjected to 7-day differentiation, immunostained for a neural marker (G) and counted for percentages of neuron (H) vs. astrocyte (I) differentiation. Examples of images (G) show neuronal marker Tuj1 immunostaining (red). The entire cell populations were visualized by GFP (green) and DAPI staining (blue). J-O. Three NSC lines, DNIκBα-hNSCHFD, GFP-hNSCHFD and GFP-hNSCchow, established using male C57BL/6 mice that received 4-month HFD vs. chow were subjected to Brdu labeling (J&K) and cell output assay (L), and neural differentiation (M-O) at a low passage (Passage 6). J&K: Same numbers of dissociated cells were cultured in growth medium and were pulse labeled with Brdu at Day 3. Cells in slides were stained for Brdu (red) (J) and counted for the percentage of Brdu-positive (Brdu+) cells (K). The entire populations of cells were visualized by GFP (green) and DAPI staining (blue). L: Same numbers of dissociated cells were cultured in growth medium and analyzed for cell outputs over 5 passages. M-O: Same numbers of dissociated cells were induced for 7-day differentiation, immunostained for a neural marker (M) and counted for percentages of neuron (N) vs. astrocyte (0) differentiation. Examples of images (M) show neuronal marker Tuj1 immunostaining (red). The entire cell populations were visualized by GFP (green) and DAPI staining (blue). *P<0.05, **P<0.01, n=4-6 per group (A-O), comparisons between indicated groups or between green lines and red/blue lines at the matched passages (F&L). Error bars reflect means±SEM. GFP: GFP-hNSC (red bars/lines); CAIKKβ: CAIKKβ-hNSC (green bars/lines); DNIκBα: DNIκBα-hNSC (blue bars/lines); GFPchow: GFP-hNSCchow (red bars/lines); GFPHFD: GFP-hNSCHFD (green bars/lines); DNIκBaHFD: DNIκBα-hNSCHFD (blue bars/lines). Scale bar=50 μm.

FIG. 5A-5R. Neurogenetic and metabolic effects of hNSC-specific IKKβ manipulations in mice. A-D. Mice with IKKβ knockout in nestin-positive cells were generated by crossing Nestin-Cre mice with IKKβlox/lox mice, termed Nestin/IKKβlox/lox mice. Littermate IKKβlox/lox mice were used as controls. Mice were maintained on chow vs. HFD feeding for 5-6 months since weaning and analyzed for total numbers of Sox2 positive (Sox2+) cells (A), total neurons (B), POMC neurons (C), and AGRP (D) in the arcuate nucleus, using immunostaining of Sox2, NeuN, POMC and AGRP, respectively. E-R. Adult chow-fed C57BL/6 mice received mediobasal hypothalamus injections of lentiviruses expressing CAIKKβ controlled by Sox2 promoter. Control mice received injections of lentiviruses with CAIKKβ removed. E: Mice at 2 weeks post viral injection were co-immunostained for Sox2 (green) and IκBα (red) in the mediobasal hypothalamus. F-R: Mice at ˜12 weeks post viral injection were analyzed for Sox2-positive (Sox2+) cells (F), total neurons (G), POMC neurons (H) and AGRP neurons (I) in the arcuate nucleus (ARC) via immunostaining of Sox2, NeuN, POMC and AGRP, respectively. J-R: Mice were profiled for food intake (J), food intake normalized by lean bass (K), oxygen (02) consumption normalized by lean mass (L), body weight (M), lean mass vs. fat mass (N&O), area under curve (AUC) of GTT (P), and fasting blood insulin (Q) and leptin (R) levels. Data were obtained in mice at Week 11-13 (J-L, N-R) or Week 0 vs. 12 (M) post viral injection. *P<0.05, **P<0.01, ***P<10-3, n=4-6 mice per group (A-D, F-I) and 6-10 mice per group (J-R). Error bars reflect means±SEM. Scale bar=25 μm (E).

FIG. 6A-6N. In vivo hypothalamic implantation of NSCs engineered with NF-κB inhibition. A-H. DNIκBα-hNSC vs. GFP-hNSC were injected bilaterally (8,000 cells per side) into the mediobasal hypothalamus of chow-fed male C57BL/6 mice (3-4 months old). Injection of vehicle PBS (phosphate buffered saline) was included as an additional control. Following injection, mice received HFD vs. chow feeding. A&B: Longitudinal follow-up of implanted cells in the mediobasal hypothalamus of HFD-fed mice (A) and survival curves of grafted cells of HFD- vs. chow-fed mice (B) at indicated days post implantation. GFP shows the distribution of implanted cells, and DAPI staining reveals nuclei of all the cells in the sections. C&D: Representative staining of neuronal marker NeuN (C left) and POMC neuron marker α-MSH (C right) in HFD-fed mice implanted with DNIκBα-hNSC and cell counting in HFD-fed vs. chow-fed mice (D) at 30 days post implantation. NeuN staining and GFP are shown in red and green, respectively. Merged color (yellow) reflects differentiation of implanted cells into neurons or POMC neurons. Cell nuclear staining (blue) by DAPI reveals the entire populations of cells. Matched images for HFD-fed mice with implanted with GFP-hNSC had no survival of GFP-positive cells at Day 30 and were not shown. E-H: Average daily food intake (E), longitudinal body weight follow-up (F), glucose tolerance (G), and fasting insulin levels (H) of HFD-fed vs. chow-fed mice. Data in G&H were obtained from mice at Week 11-12 post implantation. Metabolic profiles of mice with GFP-hNSC injection were similar to that of vehicle PBS-injected mice (data not shown). iPS-derived NSCs engineered with DNIκBα vs. GFP were injected bilaterally (8,000 cells per side) into the mediobasal hypothalamus of chow-fed C57BL/6 mice. Following injection, mice were divided into subgroups to receive HFD vs. chow feeding. I: iPS cells and embryoid body (EB). Data show iPS cells cultured on feeder cells (left) and iPS cells-derived EB (right). J: Characterization of iPS-derived Neurospheres (NS). Data show NSC markers Sox2 and nestin in iPS-derived NS (left) and differentiation of dissociated cells into Tuj1-positive neurons (red), GFAP-positive astrocytes (green) and O4-positive oligodendrocytes (data not shown). Cell nuclear staining (blue) by DAPI reveals all cells in the slides. K-N: Average daily food intake (K), longitudinal body weight follow-up (L), glucose tolerance (M), and fasting insulin levels (N) of HFD- and chow-fed mice implanted with iPS-derived NSC expressing DNIκBα vs. GFP. Data in M&N were obtained at Week 10-11 post implantation. Body weight profiles of chow-fed mice between DNIκBα and GFP implantation were similar and not presented. *P<0.05, **P<0.01, ***P<0.001, n=4-6 mice per group (B, D), and n=8-12 mice per group (E-H, K—N). Error bars reflect means±SEM. GFP (black and red bars/curves): GFP-hNSC (B, D-H) or GFP-NSCiPS (K-N); DNIκBα (blue and green bars/curves): DNIκBα-hNSC (B, D-H) or DNIκBα-NSCiPS (K-N) in chow-fed (black and blue bars/dotted curves) vs. HFD-fed (red and green bars/solid curves) mice. Scale bar=50 μm (A, C, I) and 25 μm (J).

FIG. 7A-7K. Notch signaling links IKKβ/NF-κB to impaired hNSC and related physiology. A. CAIKKβ-hNSC, DNIκBα-hNSC and GFP-hNSC were analyzed for mRNA levels of Notch signaling components. Dl1 through Dl4: delta-like ligand 1 through 4, respectively. B. Cell models GFP-hNSCHFD and GFP-hNSCchow described in FIG. 4J-O were analyzed for protein levels of the active form of Notch 1 (cleaved fragment of Notch 1) via Western blotting. C&D. GFP-hNSCHFD (established in FIG. 4J-0) were infected with a mixed pool of Notch 1, 2, 3 and 4 shRNA lentiviruses to generate Notch shRNA-hNSCHFD. Control shRNA-hNSCHFD were generated by infecting GFP-hNSCHFD with control shRNA lentiviruses. Data demonstrate immunostaining (red) of neuronal marker Tuj1 (C) and cell counting of Tuj1-positive (Tuj1+) and GFAP-positive (GFAP+) cells (D) following 7-day differentiation. DAPI staining (blue) revealed the entire populations of cells. Staining of astrocyte marker GFAP was not presented. E. C57BL/6 mice received chow vs. HFD feeding for 4 months since weaning. Brain sections across the arcuate nucleus were co-immunostained for the active form of Notch 1 (red) and Sox2 (green). Presence of merged color (yellow) in Sox2-positive cells of HFD-fed mice but not chow-fed mice indicated Notch activation under HFD feeding condition. F-K. GFP-hNSC were co-infected with a mixed pool of shRNA lentiviruses against Notch 1, 2, 3 and 4 to generate Notch shRNA-hNSC. Control shRNA-hNSC were generated via infection with control shRNA lentiviruses. Cells were injected bilaterally (8,000 cells/side) into the mediobasal hypothalamus of chow-fed C57BL/6 mice, which then received HFD vs. chow feeding. F-G: Data show representative staining of neuronal marker NeuN (F left) and POMC neuron marker α-MSH (F right) for HFD-fed mice implanted with Notch shRNA-hNSC and cell counting for both chow-fed and HFD-fed mice (G) at 5 weeks post implantation. NeuN staining and GFP are visualized in red and green, respectively. Merged color (yellow) reflected the differentiation of grafted cells into neurons or POMC neurons. Nuclear staining (blue) by DAPI reveals the entire populations of cells. Matched images for HFD-fed mice with implanted with Control-hNSC had no survival of GFP-positive cells and were not presented. H-K: Average daily food intake (H), body weight (I), glucose tolerance (J), and fasting insulin levels (K) of HFD- and chow-fed mice. Data were obtained at Week 10-11 (H, J, K) or Week (W) 0 vs. 10 (I) post injection. Body weight profiles in chow-fed mice implanted with Notch shRNA-hNSC vs. control shRNA-hNSC were similar (data not shown). *P<0.05, **P<0.01, ***P<0.001, n=4-6 per group (A, D, G), and 8-10 mice per group (H-K). Error bars reflect means±SEM. GFP (black and red bars/curves): Con: Control shRNA-NSC (black and red bars/curves in G-K); Notch sh: Notch shRNA-NSC (blue and green bars/curves in G-K) in chow-fed (black and blue bars/dotted curves) vs. HFD-fed (red and green bars/solid curves) mice. Scale bar=50 μm (C&F) and 25 μm (E).

### DETAILED DESCRIPTION OF THE INVENTION

The methods disclosed herein are useful for treating an obese subject. In an embodiment, an “obese” subject is characterized by the subject having a body mass index of 30.0 or greater (and thus includes the states of significant obesity, morbid obesity, super obesity, and super morbid obesity). In an embodiment, in regard to gender, women with over 30% body fat are considered obese, and men with over 25% body fat are considered obese. The methods disclosed herein are also applicable to treating an overweight subject. In an embodiment, an overweight subject is one having a body mass index of from 25.0 to 29.9.

The methods disclosed herein are useful for treating an obesity comorbidity in a subject. In an embodiment, the obesity comorbidity is being treated and is diabetes, type 2 diabetes, hypertension, heart disease, or stroke. In a preferred embodiment, the obesity comorbidity is type 2 diabetes.

As used herein, to treat obesity in a subject who has obesity means to stabilize, reduce, ameliorate or eliminate a sign or symptom of obesity in the subject. As used herein, to treat an obesity comorbidity in a subject who has obesity means to stabilize, reduce, ameliorate or eliminate a sign or symptom of the obesity comorbidity in the subject.

As used herein, a neural stem cell is a stem cell derived from the central nervous system of a mammal. In an embodiment, the neural stem cell is not derived from an embryo. In an embodiment, the neural stem cell is derived from a live donor subject of the same species as the subject being treated, or from a cadaver of the same species as the subject being treated. In an embodiment, the neural stem cell is a hypothalamic stem cell. In an embodiment, the neural stem cell is derived from the subject being treated for the obesity or the obesity comorbidity. In an embodiment, the neural stem cells are human neural stem cells.

As used herein, IκB kinase β(“IKKβ”) is inhibitor of kappa light polypeptide gene enhancer in B-cells, kinase beta. In an embodiment, IκB kinase β is the enzyme encoded by NCBI Gene ID: 3551. In an embodiment, IκB kinase β is the enzyme designated as ENZYME entry: EC 2.7.11.10. In an embodiment, the IKKβ is human.

As used herein, NF-κB is nuclear factor of kappa light polypeptide gene enhancer in B-cells. In an embodiment, the NF-κB is human. In an embodiment, the NF-κB is encoded by Gene ID: 4790.

As used herein, Notch 1 is a protein encoded by a Notch 1 gene. In an embodiment, the Notch 1 is human. In an embodiment, the Notch 1 is the protein encoded by NCBI Gene ID: 4851. As used herein, Notch 2 is a protein encoded by a Notch 2 gene. In an embodiment, the Notch 2 is human. In an embodiment, the Notch 2 is the protein encoded by NCBI Gene ID: 4853. As used herein, Notch 3 is a protein encoded by a Notch 3 gene. In an embodiment, the Notch 3 is human. In an embodiment, the Notch 3 is the protein encoded by NCBI Gene ID: 4854. As used herein, Notch 4 is a protein encoded by a Notch 4 gene. In an embodiment, the Notch 4 is human. In an embodiment, the Notch 4 is the protein encoded by NCBI Gene ID: 4855.

A method is provided of treating obesity or an obesity comorbidity in a mammalian subject comprising administering to the subject an amount of an agent effective to treat obesity or the obesity comorbidity, which agent inhibits (i) IκB kinase β (IKKβ) activation of nuclear factor kappa-light-chain-enhancer of activated B cells (NF-κB) or (ii) Notch signaling, in a manner so as to permit the agent to enter the hypothalamus of the subject, so as to treat the obesity or the obesity comorbidity in the subject.

In an embodiment, the agent is an inducible pluripotent cell comprising a heterologous nucleic acid or having a genetic sequence deleted therein or a neural stem cell comprising a heterologous nucleic acid or having a genetic sequence deleted therein. In an embodiment, the inducible pluripotent cell or the neural stem cell is a human or a human-derived cell. In an embodiment, the neural stem cell is a hypothalamic stem cell. In an embodiment, the inducible pluripotent cell or neural stem cell comprises a heterologous nucleic acid encoding dominant-negative IκBα. In an embodiment, the inducible pluripotent cell or neural stem cell comprises a heterologous nucleic acid encoding a dominant-negative IκBα transfected via means of a viral vector. In an embodiment, the viral vector is lentiviral. In an embodiment, the inducible pluripotent cell or neural stem cell has a IKKβ genetic sequence deleted. In an embodiment, the IKKβ genetic sequence is a genetic sequence which encodes IKKβ. In an embodiment, the IKKβ genetic sequence deleted is a portion of the complete genetic sequence encoding IKKβ deletion of the portion is sufficient to prevent or inhibit expression of functional IKKβ. As used herein, IκBα is inhibitor of nuclear factor kappa-B kinase subunit alpha. In an embodiment, the IκBα is encoded by Gene ID: 1147. In an embodiment, the IκBα is human. In an embodiment, the IKKβ is human.

In an embodiment, the inducible pluripotent cell or neural stem cell comprises a shRNA directed against a Notch 1, Notch 2, Notch 3 or Notch 4. In an embodiment, the inducible pluripotent cell or neural stem cell comprises a shRNA directed against a Notch 1, Notch 2, Notch 3 or Notch 4 transfected via means of a viral vector. In an embodiment, the viral vector is lentiviral.

In an embodiment of the methods, the agent is administered centrally. In an embodiment of the methods, the agent is administered peripherally in a manner permitting a therapeutic amount of the agent to enter the hypothalamus of the subject.

As used herein, an “inducible pluripotent stem cell” is a cell derived from a somatic cell of a mammalian subject and induced into a pluripotent state by a method known in the art. In a preferred embodiment, the cell has been derived from the subject being treated. Cells may be induced by any method of such known in the art, e.g. involving Oct-3/4 and/or certain members of the Sox gene family (Sox1, Sox2, Sox3, and Sox15). Additional genes may be used to increase induction efficiency, e.g. Klf1, Klf2, Klf4, and Klf5, the Myc family (c-myc, L-myc, and N-myc), Nanog, and LIN28. (See, for example, Takahashi et al., Cell. (2006) August 25; 126(4):663-76; Zhou H, Wu S, Joo J Y, et al. (May 2009). “Generation of Induced Pluripotent Stem Cells Using Recombinant Proteins”. Cell Stem Cell 4 (5): 381-4; and Okita, K; Ichisaka, T; Yamanaka, S (2007). “Generation of germline-competent induced pluripotent stem cells”. Nature 448 (7151): 313-7, the content of each of which is hereby incorporated by reference).

As used herein, the term “heterologous nucleic acid,” with regard to its presence in, or introduction into, a cell refers to nucleic acid that is not naturally present in the cell, or a nucleic acid which is present in a position other than its naturally occurring position in the cell. It is understood that wherein the heterologous nucleic acid encodes a peptide, polypeptide or protein, the heterologous nucleic acid is incorporated into the cell in a fashion so as to permit the expression of the respective peptide, polypeptide or protein encoded.

As used herein, the term “vector” refers to a nucleic acid molecule capable of transporting another nucleic acid to which it has been linked. Preferred vectors are those capable of autonomous replication and/expression of nucleic acids to which they are linked. Vectors capable of directing the expression of genes to which they are operatively linked are referred to herein as “expression vectors.” In general, expression vectors of utility in recombinant DNA techniques are often in the form of “plasmids” which refer to circular double-stranded DNA that in their vector form are not bound to the chromosome. In the present specification, “plasmid” and “vector” are used interchangeably as the plasmid is the most commonly used form of vector. However, the invention is intended to include such other forms of expression vectors that serve equivalent functions. In an embodiment the heterologous nucleic acids of the present methods are introduced into a cell using a vector or expression vector. In a preferred embodiment, the vector or expression vector is a lentiviral vector.

As used herein, the term “expression,” with regard to a nucleic acid, refers to the process by which a nucleotide sequence undergoes successful transcription and, for polypeptides, translation such that detectable levels of the delivered nucleotide sequence are expressed.

The vectors of the invention may also comprise a promoter sequence. As used herein, the term “promoter” refers to the minimal nucleotide sequence sufficient to direct transcription. Promoter elements may render promoter-dependent gene expression controllable for cell-type specific, tissue specific, or inducible by external signals or agents. Such elements are usually located in the 5′ region of the gene but may also be located in the coding, non-coding or 3′ regions of the gene. The term “inducible promoter” refers to a promoter where the rate of RNA polymerase binding and initiation of transcription can be modulated by external or internal stimuli. The term “constitutive promoter” refers to a promoter where the rate of RNA polymerase binding and initiation of transcription is constant and relatively independent of external or internal stimuli. A “temporally regulated promoter” is a promoter where the rate of RNA polymerase binding and initiation of transcription is modulated at a specific time during development. A “tissue-specific” promoter favors expression of the transgene in the tissue that the promoter is specific for. The promoter sequences of the vectors of the invention may be any of the promoters described herein. In an embodiment, the heterologous nucleic acid used in a method of the present invention comprises a brain-specific or hypothalamus-specific promoter which favors expression of the transgene in subject brain, or more specifically in the subject's hypothalamus.

In an embodiment, the Notch pathway is inhibited in the hypothalamus of the subject by an siRNA (small interfering RNA). As used herein, an siRNA comprises a portion which is complementary to an mRNA sequence encoding a human Notch 1, human Notch 2, human Notch 3 or human Notch 4, and the siRNA is effective to inhibit expression of the human Notch 1, human Notch 2, human Notch 3 or human Notch 4, respectively. In an embodiment, the siRNA comprises a double-stranded portion (duplex). In an embodiment, the siRNA is 20-25 nucleotides in length. In an embodiment the siRNA comprises a 19-21 core RNA duplex with a one or 2 nucleotide 3′ overhang on, independently, either one or both strands. The siRNA can be 5′ phosphorylated or not and may be modified with any of the known modifications in the art to improve efficacy and/or resistance to nuclease degradation.

In one embodiment, a siRNA of the invention comprises a double-stranded RNA wherein one strand of the double-stranded RNA is 80, 85, 90, 95 or 100% complementary to a portion of an RNA transcript of a gene encoding a human Notch 1, human Notch 2, human Notch 3 or human Notch 4. In another embodiment, a siRNA of the invention comprises a double-stranded RNA wherein one strand of the RNA comprises a portion having a sequence the same as a portion of 18-25 consecutive nucleotides of an RNA transcript of a gene encoding a human Notch 1, human Notch 2, human Notch 3 or human Notch 4. In yet another embodiment, a siRNA of the invention comprises a double-stranded RNA wherein both strands of RNA are connected by a non-nucleotide linker. Alternately, a siRNA of the invention comprises a double-stranded RNA wherein both strands of RNA are connected by a nucleotide linker, such as a loop or stem loop structure.

In one embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length. In another embodiment, a siRNA of the invention is 40, 41, 42, 43, 44, 45, 46, 47, 48, 49, 50, 51, or 52 nucleotides in length. In yet another embodiment, a siRNA of the invention is 46 nucleotides in length.

In another embodiment, an siRNA of the invention comprises at least one 2′-sugar modification. In another embodiment, an siRNA of the invention comprises at least one nucleic acid base modification. In another embodiment, an siRNA of the invention comprises at least one phosphate backbone modification.

In one embodiment, inhibition of human Notch 1, human Notch 2, human Notch 3 or human Notch 4 in the hypothalamus is effected by a short hairpin RNA (“shRNA”). The shRNA is introduced into the cell by transduction with a vector and the cells are introduced into the subject in a manner to gain entry into the hypothalamus. In an embodiment, the vector is a lentiviral vector. In an embodiment, the vector comprises a promoter. In an embodiment, the promoter is a U6 or H1 promoter. In an embodiment the shRNA encoded by the vector is a first nucleotide sequence ranging from 19-29 nucleotides complementary to the target gene, in the present case human Notch 1, human Notch 2, human Notch 3 or human Notch 4. In an embodiment the shRNA encoded by the vector also comprises a short spacer of 4-15 nucleotides (a loop, which does not hybridize) and a 19-29 nucleotide sequence that is a reverse complement of the first nucleotide sequence. In an embodiment the siRNA resulting from intracellular processing of the shRNA has overhangs of 1 or 2 nucleotides. In an embodiment the siRNA resulting from intracellular processing of the shRNA overhangs has two 3′ overhangs. In an embodiment the overhangs are UU.

Relevant molecular techniques can be found in Sambrook, Molecular Cloning: A Laboratory Manual (Third Edition), Cold Spring Harbor Laboratory Press (CSH Press), 2001 (ISBN-10: 0879695773; ISBN-13: 978-0879695774), the contents of which are hereby incorporated by reference in their entirety.)

Other Notch inhibitors are known, such as R04929097 and DAPT. In an embodiment, the Notch inhibitor is a compound having one of the following structures, or a composition comprising such:

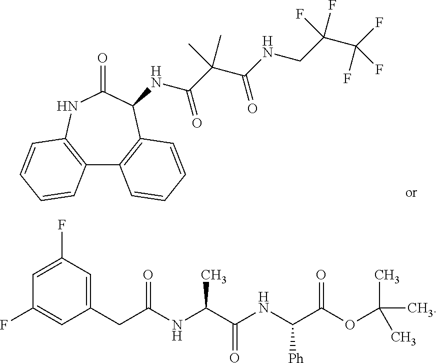

Other agents that are inhibitors of IKKβ which can be employed in the methods of the present invention are known, e.g. (2-(1-adamantyl)ethyl 4-[(2,5-dihydroxyphenyl)methylamino]benzoate), e.g. (7-[2-(cyclopropylmethoxy)-6-hydroxyphenyl]-5-[(3 S)-3-pipendinyl]-1,4-dihydro-2H-pyrido[2,3-d][1,3]oxazin-2-one hydrochloride), e.g. see Suzuki et al., Novel IκB kinase inhibitors for treatment of nuclear factor-κB-related diseases, March 2011, Vol. 20, No. 3, Pages 395-405, hereby incorporated by reference in its entirety.

The agents described herein can be administered to the subject in a pharmaceutical composition comprising a pharmaceutically acceptable carrier. The pharmaceutically acceptable carrier used can depend on the route of administration. As used herein, a “pharmaceutically acceptable carrier” is a pharmaceutically acceptable solvent, a suspending vehicle, for delivering the instant agents to the animal or human subject. The carrier may be liquid or solid and is selected with the planned manner of administration in mind. Liposomes are also a pharmaceutically acceptable carrier. Pharmaceutically acceptable carriers are known in the art, and include, but are not limited to, additive solution-3 (AS-3), saline, phosphate buffered saline, Ringer's solution, lactated Ringer's solution, Locke-Ringer's solution, Krebs Ringer's solution, Hartmann's balanced saline solution, and heparinized sodium citrate acid dextrose solution. In an embodiment the pharmaceutical carrier is acceptable for enteral or parenteral administration into the central nervous system of a mammal.

The agents can be administered together or independently in admixtures with suitable pharmaceutical diluents, extenders, excipients, or carriers (collectively referred to herein as a pharmaceutically acceptable carrier) suitably selected with respect to the intended form of administration and as consistent with conventional pharmaceutical practices.

Techniques and compositions for making dosage forms useful in the invention are described-in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Dosing can be any method or regime known in the art. For example, daily, twice daily, weekly, bi-weekly, monthly, as needed, and continuously. Implants are advantageous for continuous administration, but are not the only means of continuous administration for the present methods.

Administration can be in any manner which permits an effective amount of the agent to enter the hypothalamus of the subject. The administration may be centrally or peripherally.

Central administration may be, in non-limiting examples, in a manner which physically introduces the agent across the blood brain barrier, by an injection or via an implant (for example placed by stereotactic surgery). In non-limiting examples of central administration, the agent may be administered in an epidural manner (e.g. injection or infusion into the epidural space), in an intracerebral manner (e.g. direct injection into the brain) or intracerebroventricularly (into the cerebral ventricles)

The agent may be administered to the nasal mucosa of the subject. In an embodiment, administration to the nasal mucosa results in delivery of the agent to the central nervous system of the subject. In this regard and without being bound to any particular theory, it is believed that targeting the CNS by nasal administration is based on capture and internalization of substances by the olfactory receptor neurons, which substances are then transported inside the neuron to the olfactory bulb of the brain. Olfactory receptor neurons from the lateral olfactory tract within the olfactory bulb project to various regions such as the hypothalamus and other regions of the brain that are not directly involved in olfaction. These substances may also pass through junctions in the olfactory epithelium at the olfactory bulb and enter the subarachnoid space, which surrounds the brain, and the cerebral spinal fluid (CSF), which bathes the brain. Either pathway allows for targeted delivery without interference by the blood brain barrier, as neurons and the CSF, not the circulatory system, are involved in these transport mechanisms. Accordingly, intranasal delivery pathways permit compartmentalized delivery of compositions with substantially reduced systemic exposure and the resulting side effects. As further advantages, nasal delivery offers a noninvasive means of administration that is safe and convenient for self-medication. Intranasal administration can also provide for rapid onset of action due to rapid absorption by the nasal mucosa. This characteristic of nasal delivery result from several factors, including: (1) the nasal cavity has a relatively large surface area of about 150 cm² in man, (2) the submucosa of the lateral wall of the nasal cavity is richly supplied with vasculature, and (3) the nasal epithelium provides for a relatively high drug permeation capability due to thin single cellular layer absorption.

The agent may be administered peripherally in a manner which permits entry of the agent into the hypothalamus of the subject. In non-limiting examples, the agent is administered enterically, orally, intravenously, intramuscularly, subcutaneously, intrathecally. In an embodiment, when the agent is being administered peripherally, the subject is also administered before, during or after administration of the agent a second substance, or a therapy, which enhances movement of the agent across the blood-brain barrier (BBB) of the subject. Methods known in the art to improve permeability of the BBB include disruption by osmotic means, biochemically by the use of vasoactive substances such as bradykinin, or localized exposure to high-intensity focused ultrasound.

In accordance with the methods of the present invention, the subject is a mammal. Preferably, the subject is a human.

Also provided is a method of identifying an agent as a candidate treatment for obesity or an obesity comorbidity in a subject, the method comprising testing if the agent inhibits IKKβ/NF-κB activation by contacting the IKKβ and/or NF-κB with the agent, and determining if the agent is an inhibitor of IKKβ/NF-κB activation, wherein if the agent does not inhibit IKKβ/NF-κB activation it is not a candidate treatment, and wherein if the agent does inhibit IKKβ/NF-κB activation it is a candidate treatment.

Also provided is a method of identifying an agent as a candidate treatment for obesity or an obesity comorbidity in a subject, the method comprising testing whether the agent inhibits IKKβ/NF-κB in the hypothalamus of a non-human mammal, and determining if the agent is an inhibitor of IKKβ/NF-κB activation in the hypothalamus, wherein if the agent inhibits IKKβ/NF-κB in the hypothalamus of the non-human mammal it is a candidate treatment, and wherein if the agent does not inhibit IKKβ/NF-κB in the hypothalamus of the non-human mammal it is not a candidate treatment.

Also provided is a method of identifying an agent as a candidate treatment for obesity or an obesity comorbidity in a subject, the method comprising testing if the agent inhibits a Notch 1, Notch 2, Notch 3 or Notch 4 in the hypothalamus of a non-human mammal, and determining if the agent is an inhibitor of a Notch 1, Notch 2, Notch 3 or Notch 4 in the hypothalamus, wherein if the agent inhibits Notch 1, Notch 2, Notch 3 or Notch 4 in the hypothalamus of the non-human mammal it is a candidate treatment, and wherein if the agent does not inhibit Notch 1, Notch 2, Notch 3 or Notch 4 in the hypothalamus of the non-human mammal it is not a candidate treatment.

In embodiments of the methods, the agent is a small organic molecule of one of 1500, 1200, 1000, 800, 600 or 400 daltons or less, an RNAi molecule, a peptide, an antibody or antibody-fragment.

Also provided is a pharmaceutical composition for treating obesity or an obesity comorbidity, comprising an inducible pluripotent cell comprising a heterologous nucleic acid or having a genetic sequence deleted therein or a neural stem cell comprising a heterologous nucleic acid or having a genetic sequence deleted therein and a pharmaceutically acceptable carrier, wherein the inducible pluripotent cell or neural stem cell comprises a heterologous nucleic acid encoding a dominant-negative IκBα or comprises a dominant-negative IκBα transfected via means of a viral vector, or has a IKKβ genetic sequence deleted, or comprises a shRNA directed against a Notch 1, Notch 2, Notch 3 or Notch 4.

Also provided is an inducible pluripotent cell comprising a heterologous nucleic acid or having a genetic sequence deleted therein or a neural stem cell comprising a heterologous nucleic acid or having a genetic sequence deleted therein and a pharmaceutically acceptable carrier, wherein the inducible pluripotent cell or neural stem cell comprises a heterologous nucleic acid encoding a dominant-negative IκBα or comprises a dominant-negative IκBα transfected via means of a viral vector, or has a IKKβ genetic sequence deleted, or comprises a shRNA directed against a Notch 1, Notch 2, Notch 3 or Notch 4, for treating obesity or an obesity comorbidity.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

Herein, in the context of using NSC to treat disease (Pluchino et al., 2005; Martino and Pluchino, 2006; Wernig and Brustle, 2002; Koch et al., 2009), the biological and physiological roles of adult hypothalamic NSC, in particular in the IKKβ/NF-κB path, are investigated.

### Results

Examination of adult hypothalamic NSC (hNSC) in mice: NSC are self-renewing multi-potent cells that give rise to three lineages of neural cells including neurons, astrocytes and oligodendrocytes. Research during the recent decade has appreciated the sporadic distribution of adult NSC in the mammalian brains (Emsley et al., 2005; Mu et al., 2010; Temple, 2001). The initial identification of adult NSC was based mainly on the two brain regions which actively undergo postnatal neurogenesis, i.e., the SVZ in the forebrain (Lois and varez-Buylla, 1993) and the dentate gyrus in the hippocampus (Kuhn et al., 1996). Meanwhile, restricted numbers of adult NSCs were also detected in some other parts of the brain, including striatum and septum (Palmer et al., 1995), neocortex and optic nerve (Palmer et al., 1999), and substantia nigra (Lie et al., 2002).

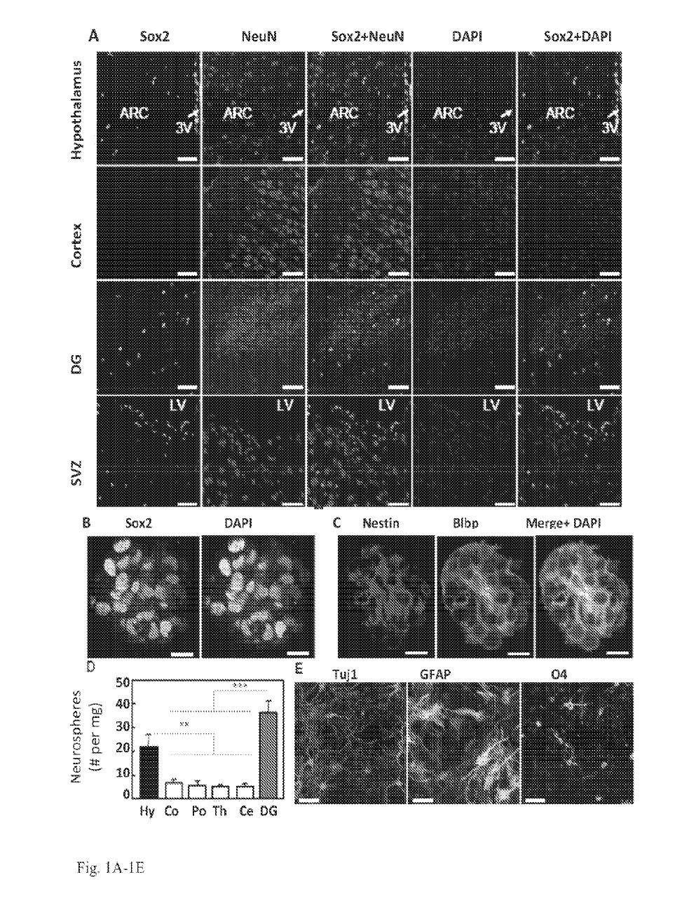

To explore the existence of adult NSC in the hypothalamus, the work in this study first analyzed the hypothalamus of adult mice for Sox2, a nuclear transcription factor of NSC which was recently proposed as the authentic NSC marker (Suh et al., 2007). Using immunostaining, it was found that Sox2-positive cells were abundantly present in the mediobasal region and the adjacent third ventricle wall of adult hypothalamus (FIG. 1A). These cells were not recognized by immunostaining of various neuronal markers such as NeuN. Other brain regions were also analyzed, and as shown in FIG. 1A, Sox2-positive cells were barely detectable in many brain regions that were examined, but were evidently present in the dentate gyrus and SVZ. Also, examined were the mediobasal hypothalamus of adult mice for Musashi-1 and nestin, two additional biomarkers for NSC and progenitors (Suh et al., 2007). Sox2-positive cells expressed both Musashi-1 and nestin (indicated by nestin promoter-directed Cre expression in Nestin-Cre mice). Thus, these data can provide an evidence to indicate the existence of adult hypothalamic NSC (hNSC) in mice.

In vitro characterization and neurogenesis of adult hNSC: Slowly-dividing NSC and their progeny (population of fast-dividing nestin-positive progenitor cells) can form neurospheres under suspension culture in medium containing growth factors EGF and b-FGF, and dissociated single cells can further form new spheres upon passages (Reynolds and Weiss, 1992; Palmer et al., 1995). In the experiments, the hypothalamus was dissected from adult mice for in vitro neurosphere formation, and it was confirmed that these neurospheres expressed all NSC markers such as Sox2, brain lipid-binding protein (Blbp), and nestin for >10 generations of passages which were followed (FIGS. 1B&C). The hypothalamus vs. various other brain regions of adult mice was examined for in vitro neurosphere-forming efficiency. As shown in FIG. 1D, the number of primary neurospheres produced by the hypothalamus was substantial compared to many other brain regions. Finally, since multi-potent neural differentiation is a hallmark of NSC, adult hypothalamic neurospheres for differentiation into various neural lineages were examined. Clearly, dissociated neurospheric cells under a 7-day differentiation procedure differentiated into neurons, astrocytes and oligodendrocytes, as assessed morphologically and using immunostaining of cell type-specific markers (FIG. 1E). Altogether, these data suggested that, in addition to SVZ and SGZ, the hypothalamus represents another critical, adult NSC-containing brain region.

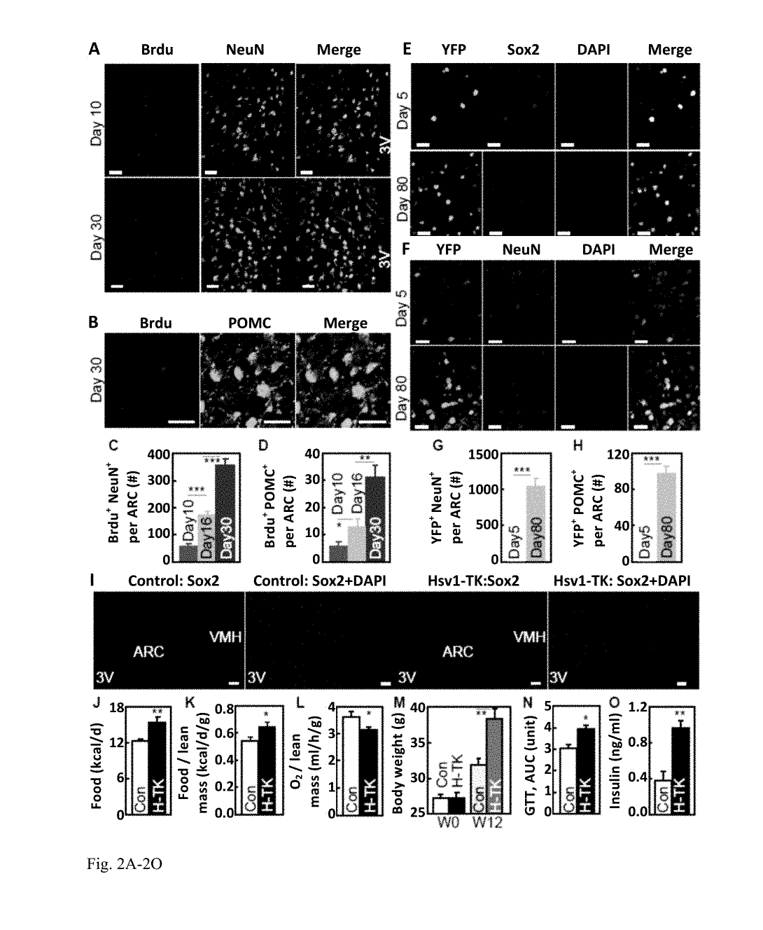

In vivo neurogenesis of adult hNSC in normal physiology: Cell labeling with 5-bromodeoxyuridine (Brdu) is an established method to track proliferating cells which has been frequently used to report the neurogenesis in adult brain (Gage, 2000; Gross, 2000). Brdu labeling was used to evaluate hNSC in adult normal mice. At 7-10 days post Brdu injection (i.p.), mice were fixed and brain sections were prepared for Brdu staining. Brdu-positive cells were confirmed evident in the mediobasal hypothalamus in addition to SVZ and dentate gyrus, but not in other brain regions (e.g., the cortex) in general. Time-course profiling of Brdu labeling was analyzed, showing that while Brdu-labeled cells usually did not express neuronal marker NeuN (FIG. 2A) but NSC marker Sox2 (data not shown) at Day 10, NeuN expression was evidently detected in a pool of Brdu-labeled cells at Day 30 post injection (FIGS. 2A&C). Co-immunostaining of Brdu with various neuropeptides further revealed that the neurogenetic activity of Brdu-labeled cells involved the neuronal subtype which expressed pro-opiomelanocortin (POMC) (FIGS. 2B&D) but not those subtypes which expressed agouti-related peptide (AGRP) or Neuropeptide Y (data not shown). Thus, these data suggested that de novo neuronal formation normally occurs in the hypothalamus of adult mice.

To further prove the existence of adult hypothalamic neurogenesis in physiology, another approach was adopted in which hypothalamic NSC were labeled with a fluorescent protein, YFP, and then longitudinally monitored if these cells underwent neuronal differentiation. To facilitate a long-term tracking, Pₛₒₓ₂-Cre lentiviruses (which expressed Cre under the control of Sox2 promoter) were injected into the mediobasal hypothalamus of ROSA-lox-STOP-lox-YFP mice. Cre-dependent removal of the STOP cassette enabled the universal promoter ROSA to induce the expression of YFP in Sox2-positive NSC cells, and importantly, expression of YFP remained in the NSC-derived neural cells despite their loss of Sox2 promoter activity. Using this tracking system, it was first confirmed that YFP was specifically expressed in Sox2-positive NSC but not NeuN-positive neurons at Day 5 post lentiviral injection (FIGS. 2E&F). Then, over an 80-day follow-up, a population of neurons was detected with YFP expression in the mediobasal hypothalamus of mice (FIGS. 2F&G). Neuropeptide immunostaining further revealed that these newly generated neurons comprised POMC neurons (FIG. 2H but not AGRP neurons (data not shown). In sum, the pharmacologic experiment (Brdu labeling) and the gene expression experiment (YFP tracking) both consistently indicated that adult hypothalamus of mice contains neuronal formation in physiology.

Mediobasal hypothalamic ablation of NSC caused metabolic disorders in mice: Since the mediobasal hypothalamus has been well recognized for the importance in controlling metabolic balance, it was tested if hNSC in the mediobasal hypothalamus has a regulatory role for metabolic physiology. To do this, the dividing Sox2-positive NSC in the mediobasal hypothalamus of adult C57BL/6 mice were specifically ablated. Hypothalamic injection of lentiviruses was performed containing Sox2 promoter-directed expression of Herpes simplex virus type-1 thymidine kinase (Hsv1-TK), a kinase which can convert nontoxic nucleoside analog ganciclovir (GCV) into a phosphorylated compound that acts as a chain terminator during DNA replication and thus specifically kills the dividing cells (Garcia et al., 2004; Tiberghien, 1998; Caruso et al., 1993; Culver et al., 1992). Following viral injection, mice were maintained on GCV-containing drinking water during the experiment to activate Hsv1-TK selectively in Sox2-positive dividing cells within the mediobasal hypothalamus. Control mice received the same procedure expect for the hypothalamic injection of control lentiviruses. Indeed, compared to controls, GCV/Hsv1-TK treatment caused a 64% reduction of Sox2-positive NSC in the mediobasal hypothalamus (FIG. 2I), leading to 11˜13% decreases in total neurons and POMC neurons in the arcuate nucleus (data not shown). It was however noted that this manipulation did not affect AGRP neurons (data not shown), indicating that hNSC have differential neurogenetic activities towards neuronal subtypes. Along with the neurogenetic defects, these mice exhibited overeating (FIGS. 2J&K) and impaired energy expenditure (FIG. 2L) despite normal chow feeding, leading to increased body weight (FIG. 2M) and adiposity (data not shown) in association with metabolic disorders including glucose intolerance (FIG. 2N), hyperinsulinemia (FIG. 2O) and hyperleptinemia (data not shown). In sum, these data indicated that the existence of adult NSC in the mediobasal hypothalamus is important for the maintenance of normal energy balance, body weight and metabolic homeostasis.

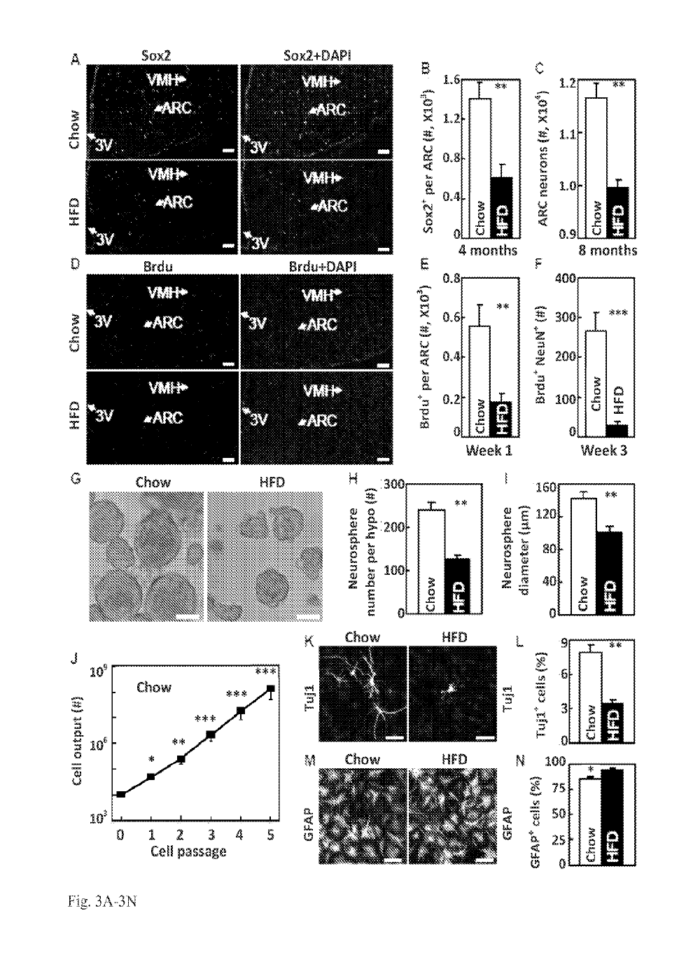

Chronic high-fat diet (HFD) feeding impaired hNSC survival and neurogenesis: Next, it was investigated if hNSC are altered in an obesogenic condition, i.e., HFD feeding, which represents a major environmental factor for obesity and co-morbidities. C57BL/6 mice were maintained under a normal chow vs. a HFD for 4 months since weaning. As expected, HFD-fed mice gradually developed obesity-diabetes syndrome, while chow-fed mice remained normal. Using Sox2 immunostaining, it was found that compared to lean mice under normal chow feeding, mice with obesity induced by 4 months of HFD feeding demonstrated a significant reduction in Sox2-positive cells in the mediobasal hypothalamus (FIGS. 3A&B). Predicted by this finding, it was observed that chronic HFD feeding (8 months) led to ˜12% decrease in total neurons (FIG. 3C) and POMC neurons (data not shown) in the arcuate nucleus. Interestingly, AGRP neurons were resistant to the effect of chronic HFD feeding (data not shown), suggesting that AGRP neurons and POMC neurons have significantly different neurogenetic characteristics. In addition, Brdu labeling was used to examine the effects of chronic HFD feeding on the neurogenetic profile in the mediobasal hypothalamus of mice. As shown in FIGS. 3D&E, cells with Brdu incorporation in the mediobasal hypothalamus were significantly fewer in HFD-fed mice compared to chow-fed mice. Time-course tracking revealed neuronal staining in some Brdu-labeled cells of chow-fed mice but rarely in HFD-fed mice (FIG. 3F). Taken together, all these data indicated that chronic HFD feeding can cause neurogenetic defects in the mediobasal hypothalamus.

Following the above in vivo analyses, in vitro experiments were performed to analyze the NSC derived from chow-fed vs. HFD-fed mice. Using an in vitro neurosphere assay, it was observed that hypothalamic neurospheres derived from HFD-fed mice were not only fewer but also smaller than that derived from chow-fed mice (FIG. 3G-I). It was also determined the proliferation rate of hNSC during 5 generations of cell passaging, starting with the same number (10⁴ cells per group) of neurospheric cells. As shown in FIG. 3J, hNSC derived from HFD-fed mice proliferated poorly, and cell outputs over 5 passages were only 7% of the control group. Further, the differentiation potential of hNSC derived from HFD-fed vs. chow-fed mice was analyzed by subjecting the same numbers of cells to 7-day differentiation. It was found that hNSC derived from HFD-fed mice displayed impaired differentiation into Tuj1-expressing neurons (FIGS. 3K&L) but enhanced differentiation into GFAP-expressing astroglial cells (FIGS. 3M&N). Such impaired proliferation and neuronal differentiation in these hNSC were observed persistently over many passages which were followed, despite the removal of HFD feeding-induced pathophysiology in cell culture condition, indicating that the disruption of adult hNSC by chronic HFD feeding was robust and at certain points difficult to be amended only by removing HFD condition.

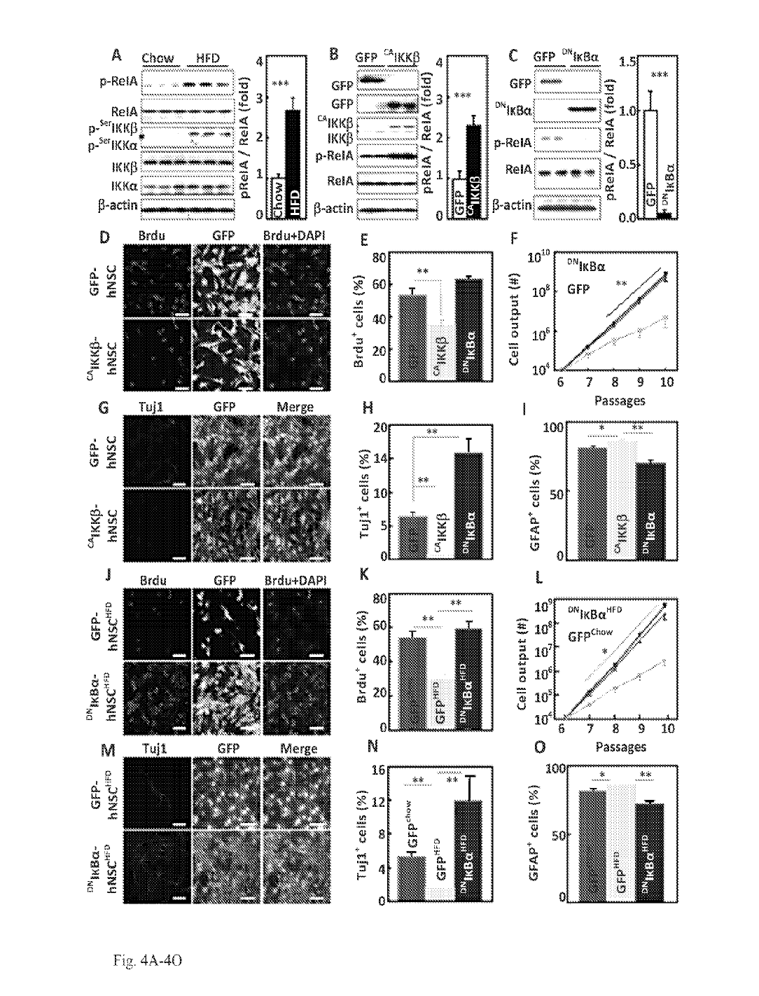

Chronic HFD feeding activated IKKβ/NF-κB in hNSC: Recent research has reported that IKKβ/NF-κB in the mediobasal hypothalamus links chronic HFD feeding to obesity development (Zhang et al., 2008; Kleinridders et al., 2009; Posey et al., 2009; Meng and Cai, 2011). This understanding provoked us to test whether adult hNSC could be affected by HFD-induced hypothalamic IKKβ/NF-κB activation. Using immunostaining of phosphorylated (Tyr199) IKKβ which reports IKKβ activation (Huang et al., 2003; Purkayastha et al., 2011a), it was found that IKKβ was activated in hypothalamic Sox2-positive cells of HFD-fed mice but not chow-fed mice (data not shown). Similar data was obtained for the hypothalamus of ob/ob mice, a genetic model which developed obesity due to leptin deficiency (data not shown). It was then directly examined if IKKβ/NF-κB signaling in hNSC that were derived from chow-fed vs. HFD-fed mice. Data revealed that phosphorylation levels of NF-κB subunit RelA (an indicator of NF-κB activation) in the hNSC derived from HFD-fed mice were significantly higher compared to chow-fed controls (FIG. 4A). Also, serine phosphorylation of IKKβ (serine residues 177 and 181) increased, which is an indispensable step for IKKβ-induced NF-κB activation (Hayden et al., 2006; Hoffmann and Baltimore, 2006; Li and Verma, 2002; Karin and Lin, 2002). Notably, IKKβ/NF-κB remained to be activated in cultured hNSC derived from HFD-fed mice, despite the absence of in vivo pathophysiology. Along with this observation, it was found that obese mice-derived hNSC produced excessive amount of TNF-α and IL-1β over several generations of cell passages (data not shown). Since TNF-α and IL-1β are not only gene products of IKKβ/NF-κB but also activators of IKKβ/NF-κB, increased release of these cytokines might contribute to sustaining IKKβ/NF-κB activation in hNSC over cell passages, although the in vivo relevance is still unclear. Also, this characteristic may not apply to early-stage obesity in which hypothalamic inflammation is reversible by caloric restriction, although it could be pathogenically relevant to late-stage obesity when involving uncompromising hypothalamic inflammation.

In vitro models of hNSC with NF-κB activation or inhibition: All the findings above guided us to hypothesize that IKKβ/NF-κB might work as a mechanistic link between obesogenic environments and defects of adult hNSC. To test this hypothesis, in vitro models were developed to directly examine the primary effects of IKKβ/NF-κB activation or inhibition on hNSC homeostasis. Using a lentiviral system to transfer DNA into the genome of infected cells (data not shown), hNSC derived from normal mice were stably transduced with cDNA encoding constitutively-active IKKβ (GFP-conjugated) to activate IKKβ/NF-κB, termed ^{CA}IKKβ-hNSC. In parallel, hNSC with stable transduction of cDNA encoding dominant-negative IκBα (GFP-conjugated) to inhibit NF-κB were generated, termed ^{DN}IκBα-hNSC. The matched control cells were hNSC with stable transduction of GFP cDNA, termed GFP-hNSC. Through antibiotic selection, only lentivirus-transduced hNSC survived and were passaged in blasticidin-containing medium, as verified by the presence of GFP in individual cells over serial passages (data not shown). According to the literature (Markakis et al., 2004), bulk culture was used rather than clonal culture, as the latter could lead to generation of biased cellular subsets. Using immunostaining, it was verified that all these hNSC models expressed NSC markers Sox2, nestin, Musashi-1 and Blbp over >15 passages which were followed up on (data not shown). Western blots confirmed that NF-κB was activated (indicated by increased RelA phosphorylation) in ^{CA}IKKβ-hNSC but inhibited (indicated by reduced RelA phosphorylation) in ^{DN}IκBα-hNSC (FIGS. 4B&C). In sum, hNSC models were generated with NF-κB activation or inhibition.

IKKβ/NF-κB activation caused defects of hNSC survival and differentiation: ^{CA}IKKβ-hNSC, ^{DN}IκBα-hNSC and GFP-hNSC were used to determine whether IKKβ/NF-κB could affect growth and proliferation of hNSC. Attached monolayer cells were pulse labeled with Brdu for 2 hours, and cells incorporated with Brdu were identified as proliferating cells. As shown in FIGS. 4D&E, the proliferation rate of ^{CA}IKKβ-hNSC decreased by ˜36% compared to the control cells. By analyzing cell numbers over 4 passages which started with the same initial number (10⁴ cells/group), the total proliferation output of ^{CA}IKKβ-hNSC was only ˜1% of GFP-hNSC (FIG. 4F). The proliferation defect of ^{CA}IKKβ-hNSC exactly reproduced the proliferation impairment of obese mice-derived hNSC (FIG. 3J). Then, a Tunnel assay was used to assess whether the growth defect of ^{CA}IKKβ hNSC was a result of enhanced apoptosis. Compared to GFP-hNSC, ^{CA}IKKβ-hNSC showed a ˜9-fold induction of Tunel-positive cells (data not shown), and this change was associated with a significantly increased entry of these cells from S/G₂ into G₀/G₁ stages (data not shown). For comparison, ^{DN}IκBα-hNSC was analyzed and it was found that ^{DN}IκBα-hNSC and GFP-NSC were relatively comparable in terms of cell proliferation rate (FIG. 4F) and the percentage of Tunel-positive cells (data not shown). To further obtain an insight into the underlying molecular basis, a host of apoptotic and anti-apoptotic genes were examined which belong to NF-κB gene targets. Apoptotic genes Bim, Bax, BNIP2, caspase-3 were substantially upregulated in ^{CA}IKKβ-hNSC and conversely down-regulated in ^{DN}IκBα-hNSC (data not shown). Upregulation of anti-apoptotic genes Bcl-2, Bcl-x1, and Traf-2 by IKKβ/NF-κB activation was also detected, but these changes were relatively less appreciable. In sum, activation of IKKβ/NF-κB in adult hNSC is predominately detrimental for cell survival.

In parallel with proliferation experiments, ^{CA}IKKβ-hNSC, ^{DN}IκBα-hNSC and control GFP-hNSC were tested for the potential of differentiation into multiple neural lineages. Cultured in the differentiation medium which did not contain growth factors, cells stopped proliferation to undergo differentiation. Data showed that ˜6% of GFP-NSC could differentiate into neurons, however, ^{CA}IKKβ-hNSC almost completely failed to differentiate into neurons, and besides, ^{DN}IκBα-hNSC differentiated into neurons more prominently than did GFP-NSC (FIGS. 4G&H). In contrast to the negative action of IKKβ/NF-κB in neuronal differentiation, differentiation of hNSC into GFAP-positive astroglial cells was however promoted in ^{CA}IKKβ-hNSC and reduced in ^{DN}IκBα-hNSC (FIG. 4I). To summarize, activation of IKKβ/NF-κB possesses a function to switch neural differentiation of hNSC from neuronal to astroglial lineage, and NF-κB inhibition can reprogram hNSC differentiation in favor of neuronal generation.

NF-κB inhibition reversed the defects of obese mice-derived hNSC: The findings above have shown that obesity condition can activate IKKβ/NF-κB in hNSC, and in the meanwhile, obesity condition and IKKβ/NF-κB activation similarly affect the survival and neuronal differentiation of hNSC. It was then tested if IKKβ/NF-κB might mediate the effect of obesity condition in inducing these defects. To do this, firstly an in vitro NSC line derived from mice was established with obesity through chronic HFD feeding, and then stably transduced these cells with dominant-negative IκBα (GFP-conjugated) vs. control GFP, using the lentiviral system (Lentiviral vectors expressing constitutively-active IKKβ (^{CA}IKKβ) dominant-negative IκBα (^{DN}IκBα) and control GFP under the control of CMV promoter. Both ^{CA}IKKβ and ^{DN}IκBα were conjugated with GFP). As such, obesity mice-derived hNSC stably expressing dominant-negative IκBα vs. GFP, termed ^{DN}IκBα-hNSC^{HFD} and GFP-hNSC^{HFD}, respectively, were generated. To provide a normal control, hNSC derived from matched chow-fed mice were stably transduced with GFP, termed GFP-hNSC^{chow}. Immunostaining verified that all these cell models over serial passages expressed NSC markers including Sox2, nestin, Musashi-1, and Blbp, as represented by images in GFP-hNSC^{HFD} (data not shown). Similar to the patterns shown in FIG. 4A-C, NF-κB activation was upregulated in GFP-hNSC^{HFD} but down-regulated in IκBα-hNSC^{HFD}, compared to GFP-hNSC^{chow}. Using these in vitro models, it was first tested if NF-κB inhibition could correct the proliferation defects of obese mice-derived hNSC. ^{DN}IκBα-hNSC^{HFD}, GFP-hNSC^{HFD}, and GFP-hNSC^{show} with the same initial cell numbers were subjected to Brdu labeling and cell output assay. Data showed that compared to the control GFP-hNSC^{chow}, GFP-hNSC^{HFD}, but not IκBα-hNSC^{HFD}, proliferated poorly in Brdu labeling (FIGS. 4J&K) and cell output analysis (FIG. 4L) experiments. Tunnel assay further revealed that apoptosis was evident in GFP-hNSC^{HFD} but not IκBα-hNSC^{HFD} (data not shown). Subsequently, neural differentiation analysis was performed and it was found that neuronal differentiation decreased while glial differentiation increased in GFP-hNSCs^{HFD}, but these differentiation defects were reversed in IκBα-hNSC^{HFD} (FIG. 4H). Hence, NF-κB inhibition can normalize the survival, proliferation and differentiation abnormalities in hNSC induced by obesity conditions (such as HFD feeding).

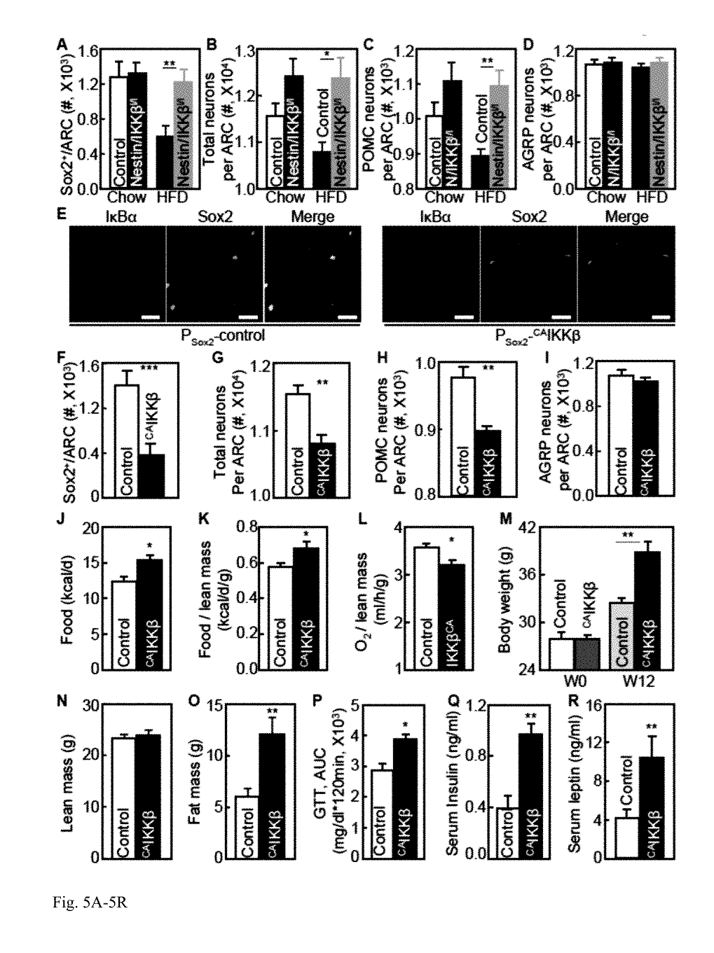

In vivo neurogenetic and metabolic effects of manipulating IKKβ/NF-κB in hNSC: To explore if IKKβ/NF-κB in NSC could have physiology/disease significance, firstly Nestin/IKKβ^{lox/lox} mice which have been previously established (Zhang et al., 2008; Meng and Cai, 2011) were used, a conditional IKKβ knockout mouse line with IKKβ gene ablated specifically in nestin-expressing cells (and the derived neural cells). Nestin/IKKβ^{lox/lox} mice were generated by crossing Nestin-Cre mice with IKKβ^{lox/lox} mice, and genotype-matched littermate IKKβ^{lox/lox} mice which had intact IKKβ gene were used as controls. Since chronic HFD feeding impaired hNSC and related neurogenesis (FIG. 3) in association with IKKβ activation (FIG. 4A), these mice were placed under chronic HFD vs. chow feeding to test if IKKβ knockout could provide an in vivo protection against the neurodegenerative effect of HFD feeding. First, it was verified that chronic (5˜6-month) HFD feeding reduced numbers in Sox2-positive cells (FIG. 5A), total neurons (FIG. 5B) and POMC neurons (FIG. 5C) but not in AGRP neurons (FIG. 5D) within the arcuate nucleus of control mice (IKKβ^{lox/lox} mice). In contrast, all these neurogenetic defects were completely prevented in Nestin/IKKβ^{lox/lox} mice (FIG. 5A-C). Thus, these data well aligned with the striking in vitro effects of NF-κB inhibition in improving survival and neuronal differentiation of NSC (FIG. 4). Such neurogenetic protection of IKKβ inhibition offered a neurogenetic mechanism which underlies the previously reported obesity-resistant phenotype in the Nestin/IKKβ^{lox/lox} mice (Zhang et al., 2008; Meng and Cai, 2011).

In addition to the loss-of-function model above, a gain-of-function model was developed with IKKβ/NF-κB activation selectively in the hNSC of the mediobasal hypothalamus. This model was generated through mediobasal hypothalamic injection of Sox2 promoter-controlled lentiviruses expressing ^{CA}IKKβ(P_{Sox2}-^{CA}IKKβ). Controls were matched mice that received mediobasal hypothalamic injection of control lentiviruses. Using IκBα degradation as an indicator of IKKβ/NF-κB activation, it was confirmed that IκBα degraded in the Sox2-positive cells but not in other hypothalamic cells of mice injected with Pₛₒₓ₂-^{CA}IKKβ(FIG. 5E). Further immunostaining revealed that ^{CA}IKKβ indeed reduced Sox2-positive cells (FIG. 5F), leading to a significant reduction in total neurons (FIG. 5G) and POMC neurons (FIG. 5H) but not AGRP neurons (FIG. 5I) in the arcuate nucleus of mice at ˜3 months post viral injection. Physiological study showed that these mice developed metabolic disorders including overeating (FIGS. 5J&K) and impaired energy expenditure (FIG. 5L). As a result, mice displayed increased weight gain (FIG. 5M) and adiposity (FIGS. 5N&O) which were associated with glucose intolerance (FIG. 5P), hyperinsulinemia (FIG. 5Q) and hyperleptinemia (FIG. 5R). In sum, loss-of-function and gain-of-function studies above both support the conclusion that IKKβ/NF-κB impairs hNSC to mediate the neurogenetic mechanism of metabolic disorders including obesity and its co-morbidities.

In vivo implantation of hNSC engineered with NF-κB inhibition: Based on the important effects of IKKβ/NF-κB in adult hNSC revealed both in vitro (FIG. 4) and in vivo (FIG. 5), a notion was conceived that this understanding could lead to in vivo application. Since mice under obesogenic (e.g., chronic HFD feeding) conditions suffered from hNSC defects (FIG. 3), it was explored if cell therapy using hNSC engineered with NF-κB inhibition could be useful. To do this, ^{DN}IκBα-hNSC vs. control GFP-hNSC established in data not shown were implanted into the hypothalamus of adult mice under the condition of HFD-induced obesity. Matched chow-fed mice receiving the same implantation were included to provide a normal reference. Since the mediobasal hypothalamus was revealed as the region enriched with hNSC (FIG. 1), this subregion was targeted in the implantation experiments, GFP-positive hNSC were delivered specifically into the middle portion of the mediobasal hypothalamus of mice. With success, hNSC were also intravenously delivered into the mediobasal hypothalamus of mice which were induced to have the blood-brain barrier (BBB) leakage (data not shown).

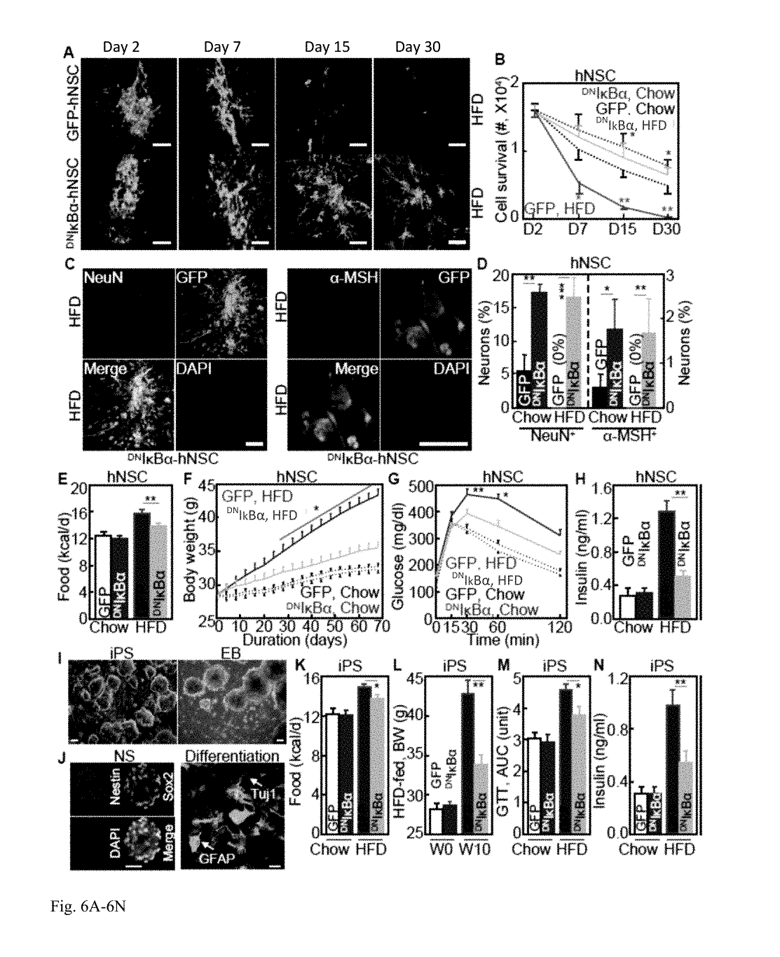

In this study, characterizing the brains of the mice with hypothalamic implantations of GFP-hNSC vs. ^{DN}IκBα-hNSC under chow or HFD feeding was focused upon. First of all, data were obtained (FIGS. 6A&B) showing that ˜30% of injected GFP-hNSC survived over a 30-day monitoring in chow-fed mice; however, injected GFP-NSC survived very poorly under HFD feeding condition which decreased by 67% at Day 7 and became rarely detectable at Day 15-30. Compared to GFP-NSC, survival of injected ^{DN}IκBα-hNSC was improved even under chow feeding, and ˜52% implanted cells remained in the hypothalamus of HFD-fed mice at Day 30 (FIGS. 6A&B). These tissue samples were subjected to immunostaining of Ki67, a cell proliferation marker, and indeed, ^{DN}IκBα-hNSC were proliferative more actively than GFP-NSC in mice, and this improvement was resistant to the anti-survival effect of HFD feeding condition (data not shown). All these data indicate that NF-κB inhibition can help hNSC to survive from chronic HFD feeding conditions.

In addition to the increased ability to survive, it was noted with interest that a significant pool of injected ^{DN}IκBα-hNSC gradually generated neural branches and even migrated from the injection site towards the surrounding regions. Immunostaining showed that while >50% of ^{DN}IκBα-hNSC maintained stem cell identity (data not shown), ˜16% of cells differentiated into NeuN-positive neurons (FIG. 6C left & 6D) at Day 30-60 post implantation. Compared to GFP-NSC, injected ^{DN}IκBα-hNSC displayed enhanced potential for neuronal differentiation in chow-fed mice and were completely resistant to the neurodegenerative effect of HFD feeding (FIG. 6D). In addition to neurons, a population of GFAP-positive astrocytes was detected differentiated by ^{DN}IκBα-hNSC (s data not shown). To prove that the grafted cells visualized by GFP fluorescence were not a result of fusion with the endogenous cells of the host animals, GFP-expressing NSC implantation into a transgenic report mouse line which globally expressed red florescent protein DsRed was performed. The experiments verified that neither GFP-expressing grafted NSC (data not shown) nor the derived neurons (data not shown) were fused with DsRed-expressing host cells. Subsequently, given that the defect of hypothalamic neurogenesis under HFD feeding significantly involved POMC neurons (data not shown), immunostaining was performed for the functional product of POMC, i.e., a melanocyte-stimulating hormone (α-MSH), and found that 1.7% of surviving ^{DN}IκBα-hNSC differentiated into POMC neurons (FIG. 6C right & 6D). On the other hand, immunostaining for other neuropeptides including AGRP and NPY did not yield positive staining in neurons differentiated by ^{DN}IκBα-hNSC. Taken together, hNSC engineered with NF-κB inhibition can differentiate into neurons under in vivo condition.

Implantation of hNSC with NF-κB inhibition counteracts obesity-T2D: While physiological implications of in vivo hNSC implantation could be multiple, a potential from the perspective of metabolic physiology was explored. Physiological experiments were performed to assess whether implantation of ^{DN}IκBα-hNSC provided benefits against HFD feeding-induced metabolic disorders. Matched chow-fed mice received the same implantation were included for comparison. Compared to GFP-NSC, implantation of ^{DN}IκBα-hNSC, despite the enhanced neurogenesis (FIG. 6D), did not alter the normal metabolic profile of chow-fed mice in terms of feeding, energy expenditure, body weight, body composition, and blood levels of glucose, insulin and leptin, as shown in FIG. 6E-H. in contrast, implantation of ^{DN}IκBα-hNSC, but not GFP-hNSC, prevented HFD feeding from inducing energy imbalance (FIG. 6E), obesity (data not shown), glucose intolerance (FIG. 6G), hyperinsulinemia (FIG. 6H) and hyperleptinemia (data not shown). In exploring the underlying mechanism, it was found that ^{DN}IκBα-hNSC prevented HFD feeding from decreasing hypothalamic POMC mRNA (data not shown), which was consistent with increased numbers of POMC neurons (FIG. 6D)—a group of arcuate neurons that critically regulate energy balance and prevent obesity development. Since POMC neurons importantly employ insulin and leptin signaling to control feeding and body weight balance, it was investigated if hypothalamic insulin resistance and leptin resistance, two key factors in the central mechanism of obesity and T2D, could be prevented by NSC implantation. Mice with implantation of ^{DN}IκBα-hNSC but not GFP-hNSC were protected from the effects of HFD feeding in impairing the central actions of insulin and leptin in controlling feeding. Using immunostaining, it was further verified that a fraction of neurons derived from ^{DN}IκBα-hNSC responded to leptin (data not shown) and insulin (data not shown), further supporting the neurogenetic basis for the observed anti-disease effect. Meanwhile, it was also excluded any non-specific effect related to inflammatory changes due to the injection procedure, as the injection per se caused neither IKKβ activation (data not shown) nor induction of cytokine expression (data not shown) in the hypothalamus of mice following the post-injection recovery. To summarize, this implantation study can point to a potential strategy for obesity and T2D intervention through targeting NSC in the hypothalamus, and also indicate that IKKβ/NF-κB inhibition is critical for the success of this interventional avenue.

Implantation of iPS-derived NSC with NF-κB inhibition counteracts obesity-T2D: It was also examined if there might be alternative cell sources rather than endogenous hNSC for the implantation strategy described above. The investigation has initially assessed the possible use of inducible pluripotent stem cells (iPS), a cell model developed during recent years (Takahashi and Yamanaka, 2006). Using the protocol established in the literature (Okada et al., 2004), NSC were successfully induced from a line of mouse iPS (FIG. 6I), verified by the presence of multiple NSC markers (FIG. 6J left) and the abilities to differentiate into three neural lineages including neurons and astrocytes (FIG. 6J right) and oligodendrocytes (data not shown). Subsequently, iPS-derived NSC were generated with lentiviral expression of ^{DN}IκBα (GFP-conjugated) vs. control GFP, termed ^{DN}IκBα-NSC^{iPS}, and GFP-NSC^{iPS}, respectively. These cells were implanted into the mediobasal hypothalamus of HFD-fed vs. chow-fed mice, as described above. First, it was found that just like ^{DN}IκBα-hNSC shown in FIG. 6A-D, ^{DN}IκBα-NSC^{iPS} survived and differentiated into neurons significantly which were resistant to the neurodegenerative effects of HFD feeding. Physiological studies revealed that ^{DN}IκBα-NSC^{iPS}, but not GFP-NSC^{iPS}, significantly prevented HFD feeding from causing energy imbalance (FIG. 6K), obesity (FIG. 6L) and the disorders of glucose (FIG. 6M), insulin (FIG. 6N) and leptin (data not shown). On the other hand, Injection of neither ^{DN}IκBα-NSC^{iPS} nor GFP-NSC^{iPS} affected the normal metabolic profiles of chow-fed mice (FIG. 6K-N). In sum, iPS-induced NSC and endogenous NSC possess similar application values in treating obesity-T2D.

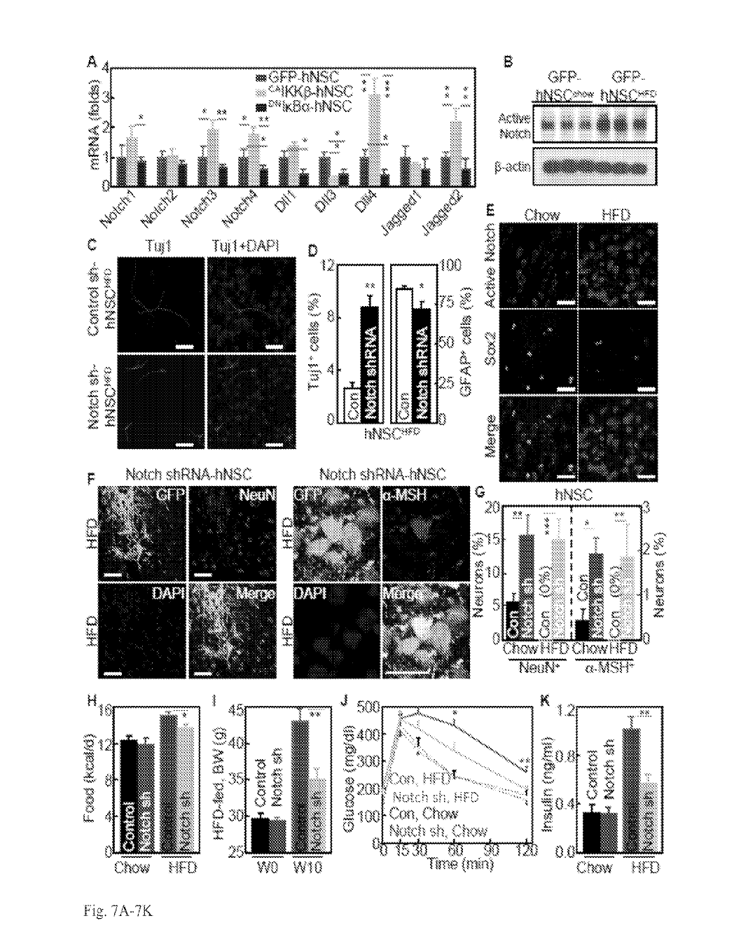

Notch signaling mediates neurogenetic defects of NSC induced by IKKβ or HFD: Finally, the potential downstream mediator for the effects of IKKβ/NF-κB in hNSC was explored. Through gene expression screening, it was observed that many components of the Notch signaling pathway, such as Notch 3 and 4, Notch protein ligands including delta-like ligand (Dll) 1 and 4 and Jagged 2, were all upregulated in ^{CA}IKKβ-hNSC and downregulated in ^{DN}IκBα-hNSC (FIG. 7A). These observations captured attention, because recent research has revealed that Notch signaling can promote apoptosis to reduce cell survival of NSC (Yang et al., 2004). More notably, Notch signaling was found to switch neural differentiation program by inhibiting neurogenesis but promoting gliogenesis, and Notch inhibition can enhance neuronal differentiation (rtavanis-Tsakonas et al., 1999; Lutolf et al., 2002; Louvi and rtavanis-Tsakonas, 2006; Carlen et al., 2009; Oya et al., 2009; Borghese et al., 2010). In this context, it was questioned whether Notch signaling pathway might mechanistically mediate the effects of IKKβ/NF-κB in hNSC. To examine this question, Notch signaling in ^{CA}IKKβ-hNSC was inhibited through co-infection with 4 types of shRNA lentiviruses that each carried a Notch isoform (Notch 1-4) shRNA. Indeed, inhibition of Notch pathway by Notch 1-4 shRNA lentiviruses was substantial, as the active (cleaved) form of Notch proteins was barely detected in hNSC by Western blot (data not shown). Importantly, it was found that Notch 1-4 shRNA lentiviruses significantly reversed the differentiation defect (data not shown) and also improved survival in ^{CA}IKKβ-hNSC. Also, it was tested if Notch inhibition could similarly reverse these defects displayed in obese mice-derived hNSC, since obese mice-derived hNSC indeed were characterized by increased activation of Notch signaling (FIG. 7B). To test this question, GFP-hNSC^{HFD} were co-infected with Notch 1-4 shRNA lentiviruses vs. control shRNA lentiviruses, and obtained data showing that Notch inhibition corrected the differentiation defect (FIGS. 7C&D) and also ameliorated survival in GFP-hNSC^{HFD}. Altogether, data suggest that Notch signaling significantly mediates the abnormalities of hNSC induced by IKKβ/NF-κB activation or chronic HFD feeding.

In vivo implantation of hNSC with Notch inhibition counteracts obesity-T2D: Hypothalamic hNSC implantation was performed to investigate whether Notch inhibition could mimic IKKβ/NF-κB inhibition in counteracting obesity and related metabolic disorders. This investigation was also rationalized by the data showing that Notch signaling was robust (indicated by the active form of Notch 1 protein) in Sox2-positie cells of HFD-fed mice but not chow-fed mice (FIG. 7E). In the experiment, C57BL/6 mice received mediobasal hypothalamic injections of GFP-hNSC which carried Notch 1-4 shRNAs vs. control shRNA, and were then maintained on HFD vs. chow feeding. Pretty much similar to NF-κB inhibition, Notch inhibition improved survival (data not shown) and neuronal differentiation (FIGS. 7F&G) of grafted hSNC in the hypothalamus of mice. Also, Notch inhibition promoted the production of POMC neurons, as revealed by α-MSH immunostaining (FIGS. 7F&G) and POMC mRNA analysis (data not shown). Physiological studies further demonstrated that implantation of Notch shRNA-hNSC, rather than control-hNSC, protected HFD-fed mice from developing energy imbalance (FIG. 7H), obesity (FIG. 7I) and the disorders of glucose (FIG. 7J), insulin (FIG. 7K) and leptin (data not shown). On the other hand, implantation of neither Notch shRNA-hNSC nor control-hNSC affected the normal metabolic profiles of chow-fed mice (FIG. 7H-K). Thus, inhibition of Notch signaling can recapitulate the effect of NF-κB inhibition in the NSC implantation with respect to counteraction against HFD-induced obesity and metabolic disorders. Finally, hNSC implantation was employed to assess the IKKβ/NF-κB-Notch connection which was established in vitro. In the experiment, hypothalamic implantation of ^{DN}IκBα-hNSC vs. GFP-hNSC was performed into mice which had already developed obesity via chronic HFD feeding, and in the meanwhile, hypothalamic Notch signaling was activated of these mice via daily third-ventricle injection of Notch protein ligand DLL4. Data revealed that DLL4 significantly abolished the anti-obesity effect of ^{DN}IκBα-hNSC implantation. Notably, this physiological effect was accompanied by the reversal of ^{DN}IκBα-hNSC induced neurogenesis (data not shown) but not an induction of hypothalamic inflammation (data not shown). Therefore, NSC implantation can, at least, primarily employ neurogenetic program to counteract against metabolic disease, although other associated factors (such as inflammatory changes) probably also participate primarily or secondarily. To summarize, hypothalamic implantation of NSC engineered with IKKβ/NF-κB or Notch inhibition possesses consistent values in preventing and treating obesity and related metabolic diseases.

### Discussion

Adult NSC direct hypothalamic neurogenesis and functions in physiology: Adult NSC belong to a small population of cells with slowly dividing rate in the brain (Morshead et al., 1994), and because of this feature, the biological and physiological functions of these cells have not been adequately appreciated despite that their existence was recognized since 1990s. It is also noted that the understandings on adult NSC to date were mainly obtained from two brain regions which have relatively active postnatal neurogenesis, i.e., SVZ and SGZ (Gage, 2000; Gross, 2000; Morrison, 2001; Temple, 2001; varez-Buylla and Lim, 2004; Gould, 2007; Whitman and Greer, 2009). Although a few in vitro and in vivo studies have recently pointed to the hypothalamus as another brain source of adult NSC (Markakis et al., 2004; Kokoeva et al., 2005; Pierce and Xu, 2010), the question remains regarding whether NSC in the hypothalamus have an important role in physiology or disease. In this work, it was found that adult NSC are abundantly present in the mediobasal hypothalamus, which is the hypothalamic region with critical functions in regulating metabolic physiology. These cells can be isolated and maintained in vitro with full characteristic of self-renewal and multi-potent differentiation into three neural lineages including neurons, astrocytes and oligodendrocytes. Using two in vivo tracking approaches, it was shown that adult NSC contributed to the hypothalamic neurogenesis including formation of neurons in mice under physiological conditions. Also, through site-specific ablation of NSC in the mediobasal hypothalamus of normal mice, it was found that these animals developed a cluster of metabolic dysfunctions including overeating, defective energy expenditure, excessive weight gain, and whole-body glucose intolerance. These findings, while being in alignment with the central action of mediobasal hypothalamus in controlling metabolic physiology, indicate an underlying but previously unknown in vivo basis that is mediated by NSC-directed neurogenesis.

Defect of adult NSC in the hypothalamus links obesogenic conditions to disease: In parallel with physiological studies, the possible involvement of hNSC was investigated in the naturally-occurring metabolic diseases, especially obesity and T2D which easily occur under chronic obesogenic environment (such as chronic nutritional excess mimicked by HFD feeding). It was found that hNSC in animals with chronic HFD feeding had severe problems of survival and neuronal differentiation, which are detectable under both in vivo and in vitro conditions. Thus, in conjunction with recent observations showing the induction of hypothalamic neurogenesis in response to pathological stimuli (Kokoeva et al., 2005; Pierce and Xu, 2010), it is likely that adult NSC in the hypothalamus possess an important function of using neurogenetic adaptation to overcome certain environmental challenges. However, this function of NSC in the mediobasal hypothalamus is impaired under chronic conditions of caloric excess such as HFD feeding. Over the time, such persistent damages in adult NSC can lead to neuronal loss to certain degree and in particular the reduction of POMC neurons which are known to be important for the control of normal energy balance. As a result, the hypothalamus cannot sufficiently employ neurogenetic process to tackle the adverse effects of chronic caloric excess. More prospectively, by extending the phenomenon that obesity is indeed frequently coupled with neurodegenerative diseases (e.g., Alzheimer's disease, Parkinson disease), the findings indicate that obesity itself can represent a subject of neural degeneration, and loss of hypothalamic plasticity due to the impairment of adult NSC can undermine central metabolic regulation to cause metabolic disease.

Hypothalamic inflammation disrupts adult NSC via IKKβ/NF-κB to cause disease. Obesity is distinctively characterized by the presence of chronic inflammation (Ruan and Lodish, 2004; Hotamisligil, 2006; Shoelson and Goldfine, 2009; Cai, 2009). Recent work revealed that IKKβ/NF-κB critically mediates obesity-associated inflammation in the periphery (Cai et al., 2005; Cai et al., 2004) and the hypothalamus (Zhang et al., 2008; Purkayastha et al., 2011b). In this study, it was found that IKKβ/NF-κB in adult NSC of the hypothalamus was normally inactive, but became highly active in response to the chronic challenge of obesogenic environment. Importantly, activation of IKKβ/NF-κB through lentiviral-mediated gene delivery reproduced the defects of adult NSC derived from animals suffered from chronic obesogenic environment. These effects are generally in agreement with the literature showing that inflammatory cytokines IL-1β and IL-6 inhibit hippocampal neurogenesis (Koo and Duman, 2008; Vallieres et al., 2002). Taken together, obesity-associated hypothalamic inflammation not only impairs the functions of matured neurons which it was previously reported (Zhang et al., 2008; Purkayastha et al., 2011b), but also deteriorates adult NSC of the hypothalamus. Hypothalamic inflammation may constitute a hostile environment for stem cell “niche” which disrupts the normal cell biology of adult NSC. In this context, an array of observations suggest IKKβ/NF-κB inhibition can reverse both survival and differentiation defects of NSC imposed by chronic obesity condition. Physiological studies further demonstrated that adult NSC-specific IKKβ/NF-κB activation in the mediobasal hypothalamus was obesogenic. Agreeably, genetic inhibition of IKKβ/NF-κB which primarily targeted nestin-expressing cells prevented HFD feeding from causing neurogenetic defects, providing a new mechanism for the anti-obesity and T2D phenotypes of these mice which was reported previously (Zhang et al., 2008; Meng and Cai, 2011). Altogether, these findings suggest that the central mechanism of obesity-T2D via hypothalamic inflammation involves a neurodegenerative basis.

Notch pathway mediates action of IKKβ/NF-κB in hNSC to underlie obesity-T2D. In addition to being an inflammatory regulator, IKKβ/NF-κB can control cell proliferation and differentiation. In terms of cell survival, depending on cell types and conditions, NF-κB can be anti-apoptotic (Hayden et al., 2006; Hoffmann and Baltimore, 2006; Li and Verma, 2002; Karin and Lin, 2002) and pro-apoptotic (Chen et al., 2011; Vousden, 2009; Dutta et al., 2006; Ryan et al., 2000; Lin et al., 1998; Qin et al., 1998). In this work, it was found that action of IKKβ/NF-κB in the hypothalamus was detrimental for both survival and neuronal generation of NSC. Also, with interest, it was found that IKKβ/NF-κB activation in hNSC inhibited neuronal differentiation in exchange for glial differentiation. Further, Notch signaling pathway was revealed to significantly account for these deleterious effects of IKKβ/NF-κB in NSC. These findings are in line with recent understandings showing that NF-κB closely interacts with Notch signaling in peripheral cells (Vacca et al., 2006; Oakley et al., 2003; Espinosa et al., 2003; Cheng et al., 2001). In general, the IKKβ/NF-κB-Notch connection well agrees with the literature showing that Notch activation can induce NSC apoptosis (Yang et al., 2004) and switch neural development from neurogenesis to gliogenesis (Borghese et al., 2010; Carlen et al., 2009; Louvi and rtavanis-Tsakonas, 2006; Oya et al., 2009; Lutolf et al., 2002; rtavanis-Tsakonas et al., 1999). Hence, Notch signaling works as a molecular pathway that mediates IKKβ/NF-κB to disrupt hNSC, and thus represents a downstream target for reversing related cell biological problems.

Engineered NSC counteract obesity-T2D under obesogenic conditions. Recent appreciation on NSC has begun to promote the therapeutic interests of using these cells, and this inspiration has already led to progresses based on a few classical neurological diseases (Pluchino et al., 2005; Martino and Pluchino, 2006; Wernig and Brustle, 2002; Koch et al., 2009; Lindvall and Kokaia, 2006). Since complex metabolic diseases like obesity and T2D have been increasingly recognized to be significantly neurogenic (Niswender et al., 2004; Munzberg and Myers, Jr., 2005; Flier, 2006; Coll et al., 2007), a neural cell therapy might provide unexpected benefits, especially considering that cell therapy could lead to a comprehensive remedy which may not be readily offered by a drug. In this work, the treatment of obesity-T2D via hypothalamic implantation of NSC which were pre-engineered with NF-κB suppression was targeted. Indeed, NF-κB inhibition is not only necessary for long-term cell survival but also in favor of neuronal differentiation. Importantly, such cell therapy significantly ameliorated obesity, insulin resistance and glucose intolerance in mice despite the obesogenic environment. In terms of the underlying mechanism, while neurogenesis is primarily involved, additional benefits might include the anti-inflammatory effects of NSC suggested by the literature (Pluchino et al., 2005; Martino and Pluchino, 2006).

### Materials and Methods

Animal models and Phenotyping. C57BL/6 mice were obtained from Jackson Laboratory. Nestin/IKKβ^{lox/lox} mice were generated by breeding Nestin-Cre mice with IKKβ^{lox/lox} mice (both lines were maintained on C57BL/6 background for >15 generations), as described previously (Zhang et al., 2008; Meng and Cai, 2011). All mice were housed in standard conditions. High-fat diet was obtained from Research Diets, Inc. Body weight of individually housed mice was measured twice per week and food intake was recorded daily. MRI measurement of lean vs. fat mass and metabolic chamber measurement of O₂ consumption were performed at the core facility at Albert Einstein College of Medicine. O₂ consumption of mice was normalized by lean body mass obtained at the same time. For GTT, overnight fasted mice were injected with glucose (2 g/kg body weight) intraperitoneally, and blood glucose levels at various time points were measured using a Glucometer (Bayer). All procedures were approved by the Institutional Animal Care and Use Committee of Albert Einstein College of Medicine.

Lentiviruses and mediobasal hypothalamic injections. The cDNAs for ^{CA}IKKβ, ^{DN}IκBα, Cre and GFP have been described previously (Zhang et al., 2008; Purkayastha et al., 2011b; Zhang et al., 2011). The cDNA for Hsv-1 TK was obtained from Addgene. Using ViraPower pLenti6.2N5 lentiviral expression system (Invitrogen), CMV promoter-controlled lentiviral vectors were constructed to direct the expression of ^{CA}IKKβ (GFP-conjugated), ^{DN}IκBα (GFP-conjugated) or GFP. Using a Sox2 promoter-controlled lentiviral system (kindly provided by F. Gage), Sox2 promoter-controlled lentiviral vectors were constructed to direct the expression of Cre, Hsv-1 TK, ^{CA}IKKβ, or control GFP. Lentiviral vectors with shRNA against Notch 1, 2, 3, 4 or matched control shRNA were purchased from Sigma. Lentiviruses were produced by co-transfecting viral expression vectors with the package plasmids into HEK 293 FT cells, as described previously (Zhang et al., 2008; Zhang et al., 2011). Bilateral injections of mediobasal hypothalamus were described previously (Zhang et al., 2008; Purkayastha et al., 2011b; Zhang et al., 2011). Briefly, anaesthetized mice under an ultra-precise stereotax (resolution: 10 μm, David Kopf Instruments) were injected with purified lentiviruses in the vehicle (PBS) into each side of the mediobasal hypothalamus through a guide cannula directed to the coordinates at 0.17 mm posterior to the bregma, 0.03 mm lateral to the middle line, and 0.50 mm below the skull surface of mice.

Chemical administration. Brdu labeling: Mice were i.p. injected with Brdu (Sigma) at 100 mg/kg body weight. Each mouse received one injection per day for 7 consecutive days. Mice were perfused with 4% PFA at indicated days post injections, and brains were removed, post-fixed and sectioned for Brdu staining. GCV/Hsv1-TK induced hNSC ablation: C57BL/6 mice were bilaterally injected in the mediobasal hypothalamus with Sox2 promoter-controlled lentiviruses carrying Hsv1-TK or control GFP. Following lentiviral injection, mice were then maintained on drinking water containing 1 mg/ml GCV (US Biological) until the end of the study.

Adult hypothalamus-derived neurospheres and analyses. Hypothalamus was dissected from adult mice as described previously (Zhang et al., 2008; Purkayastha et al., 2011b; Zhang et al., 2011). Tissues were cut into small pieces (˜1 mm³), digested with 0.25% Papain (Worthington) for 30 min at 30° C., and gently triturated for approximately 10 times using fire-polished tips. Desired cell population was separated by density gradient centrifugation. After washing with Hibernate-A medium (BrainBits LLC) twice, cells were suspended in the growth medium containing Neurobasal-A (Invitrogen), 2% B27 (Invitrogen), 10 ng/ml EGF (Sigma) and 10 ng/ml b-FGF (Invitrogen), seeded in Ultra-low adhesion 6-well plates (Corning) at a density of 10⁵ cells per well, and incubated in 5% CO₂ at 37° C. On day 7, neurospheres were collected through centrifugation, dissociated into single cells by trypsinization using TrypLE™ express media (Invitrogen), and passaged in suspension culture to form subsequent generations of neurospheres. Neurosphere counting: neurospheres prepared in 24-well plates were counted under a microscope. Neurosphere size quantitation: neurospheres were photographed microscopically and the diameters were measured using software Image J.

Adult hypothalamus-derived NSC models. Adult hypothalamus-derived neurospheric cells were maintained in EGF/b-FGF-containing growth medium. Cells at low passages were infected with various lentiviruses (containing fluorescent marker GFP) for 3 days, and followed by cell selection process through adding nucleoside antibiotic blasticidin (1 μg/ml) to the culture medium. Cells transduced with lentiviral DNAs were resistant to blasticidin, and were monitored for the induction of fluorescent protein GFP via a fluorescent microscope. Transduced cells were stably passaged in blasticidin-containing selection growth medium, and the presence of GFP in all cells was monitored over passages.

Pluripotent stem cells (iPS)-derived NSC models. Stemgent® Mouse Primary iPS Cells-NNeo was purchased from STMGENT Company. Maintenance of iPS used standard embryonic stem cells culture conditions. Briefly, the irradiated mouse embryonic fibroblasts were plated at a density of 2.5×10⁴ cells/cm² as feeder cells in gelatin-coated 6-well plates, and iPS were maintained on the feeder cells with standard embryonic stem cells culture medium containing knock-out DMEM, 10% knock-out seurm, 2 mM L-Glutamine, 0.1 mM nonessential amino acids, 1 mM sodium pyruvate, 0.1 mM β-mercaptoethanol and 10 ng/ml LIF (all from Invitrogen). For embryoid body (EB) formation, dissociated iPS cells (via 0.05% trypsin-EDTA solution) were cultured at 5×10⁵ cells/ml in the EB formation medium (which is the standard embryonic stem cells culture medium without adding LIF). Following 3 days of EB formation, cultured cells were stimulated with 5 μM RA (Sigma) for 7 days with culture medium changed every day. At Day 11, EBs were dissociated into single cells and transfer to NSC culture medium (the growth medium described above). Spheres formed after 2 passages of culture were mainly neurospheres, as confirmed by immunostaining of NSC markers, and were also examined for multi-potent differentiation into 3 neural cell lineages. To generate iPS-derived NSC with stable gene manipulation, dissociated neurospheric cells were infected with various lentiviruses (containing fluorescent marker GFP) and selected over passages using the blasticidin-containing selection medium as described above.

Cell proliferation and differentiation assays. NSC proliferation output assay: Neurospheres were dissociated into single cells and plated in Ultra-low adhesion 6-well culture plate at the density of 10⁴ cells in 1 ml of the growth medium. Cells were passaged every 5 days at a density of 10⁴ cells in 1 ml of growth medium. Viable cells in each passage were evaluated by trypan blue staining. The accumulated total cell number for each passage was calculated by assumption that the total cells from the previous passage were replated. NSC differentiation: Dissociated single cells were seeded in poly-D-lysine and laminin-coated coverslips placed in 24-well plates. Cells were cultured in the differentiation medium containing Neurobasal-A, 2% B27 and 1% FBS (all purchased from Invitrogen). Culture medium was changed every other day, and neural differentiation was induced for one week.

NSC implantation. Bilateral injections of mediobasal hypothalamus were previously described (Zhang et al., 2008; Purkayastha et al., 2011b; Zhang et al., 2011). Briefly, under an ultra-precise stereotax, a total number of 8,000 NSC was injected into each side of the mediobasal hypothalamus through guide cannula which was directed to the coordinates of 0.17 mm posterior to the bregma, 0.03 mm lateral to the middle line, and 0.50 mm below the skull surface of mice. Each mouse was monitored for post-injection recovery.

Heart perfusion, tissue/cell immunostaining, and image analysis. Mice under anesthesia were trans-heart perfused with 4% PFA, and the brains were removed, post-fixed in 4% PFA for 4 hours, and infiltrated in 20%-30% sucrose. Brain sections (20 μm) were made using a cryostat at −20° C. Cultured cells on coverslips were fixed with 4% PFA for 10 min at room temperature. For Brdu staining, samples were pre-treated with 2M HCl for 20 min followed by 2-min incubation with 0.1M sodium borate (pH 8.5). Fixed tissue sections/cells were blocked with serum of appropriate species, penetrated with 0.2% Triton-X 100, treated with primary antibodies and followed by reaction with Alexa Fluor® 488 or 555 or 633 secondary antibodies (Invitrogen). Naïve IgGs of appropriate species were used as negative controls. Primary antibodies included rabbit anti-Blbp, anti-activated Notch 1 and anti-phosphorylated (Tyr199) IKKβ antibodies (ABcam), rabbit anti-Musashil antibody (Millipore), rabbit anti-IκBα (Santa Cruz), rabbit anti-POMC (Phoenix Pharmaceuticals), mouse anti-Tuj1 and anti-Brdu antibodies (Cell Signaling), mouse anti-GFAP, anti-04, anti-nestin, anti-NeuN antibodies (Millipore), mouse anti-Sox2 antibody (R&D Systems), and sheep anti-α-MSH and guinea pig anti-AGRP antibody (Millipore). DAPI (Vector) staining revealed the nuclei of all cells in the slides. Images of immunostaining were captured under a con-focal microscope. Cell counting for hypothalamic arcuate nucleus immunostaining: serial hypothalamus sections across the arcuate nucleus were made at the thickness of single cell (10 μm), and every 5 sections were represented by one section with staining and cell counting. The numbers in representative sections were multiplied by 5 to indicate the total numbers.

Real-time RT-PCR, Western blot and biochemical assays. Total RNA was extracted from cells using TRIzol (Invitrogen) following the manual. Complementary DNA was synthesized using the Advantage RT for PCR kit (BD Biosciences). Real-time PCR was performed (triplicate reactions/sample) using the SYBR Green PCR Master Mix (Applied Biosystems). Relative gene expression levels were normalized against mRNA levels of housing-keeping gene β-actin. Western blot analysis was performed using proteins extracted from cells/tissues and dissolved in a lysis buffer. Proteins were separated by SDS/PAGE and identified by immunoblotting. Primary antibodies were rabbit anti-GFP (Sigma), anti-phosphorylated IKKα/β, anti-phosphorylated RelA, anti-IKKβ, anti-RelA, anti-cleaved Notch 1, and anti-β-actin (Cell Signaling), and anti-IKKα (Santa Cruz) antibodies. Secondary antibodies were HRP-conjugated antibodies (Pierce). TNF-α and IL-1β in cultured media were measured using ELISA kits (Ebioscience). Serum insulin and leptin were measured using insulin (Linco) and leptin (Crystal Chem. Ins) ELISA kits.

Statistical analyses: Data are presented as mean±SEM. Statistical differences were evaluated using Student's t-test for two-group comparison or ANOVA and appropriate post hoc analyses for >2-group comparisons. P<0.05 was considered significant.

### REFERENCES

Borghese, L., Dolezalova, D., Opitz, T., Haupt, S., Leinhaas, A., Steinfarz, B., Koch, P., Edenhofer, F., Hampl, A., and Brustle, O. (2010). Inhibition of Notch signaling in human embryonic stem cell-derived neural stem cells delays G1/S phase transition and accelerates neuronal differentiation in vitro and in vivo. Stem Cells 28, 955-964.
Bouret, S. G., Draper, S. J., and Simerly, R. B. (2004). Trophic action of leptin on hypothalamic neurons that regulate feeding. Science 304, 108-110.
Bouret, S. G., Gorski, J. N., Patterson, C. M., Chen, S., Levin, B. E., and Simerly, R. B. (2008). Hypothalamic neural projections are permanently disrupted in diet-induced obese rats. Cell Metab 7, 179-185.
Cai, D. (2009). NFkappaB-mediated metabolic inflammation in peripheral tissues versus central nervous system. Cell Cycle 8, 2542-2548.
Cai, D., Frantz, J. D., Tawa, N. E., Jr., Melendez, P. A., Oh, B. C., Lidov, H. G., Hasselgren, P. O., Frontera, W. R., Lee, J., Glass, D. J., and Shoelson, S. E. (2004). IKKbeta/NF-kappaB activation causes severe muscle wasting in mice. Cell 119, 285-298.
Cai, D., Yuan, M., Frantz, D. F., Melendez, P. A., Hansen, L., Lee, J., and Shoelson, S. E. (2005). Local and systemic insulin resistance resulting from hepatic activation of IKK-beta and NF-kappaB. Nat. Med. 11, 183-190.
Cameron, H. A. and McKay, R. (1998). Stem cells and neurogenesis in the adult brain. Curr. Opin. Neurobiol. 8, 677-680.
Carlen, M., Meletis, K., Goritz, C., Darsalia, V., Evergren, E., Tanigaki, K., Amendola, M., Barnabe-Heider, F., Yeung, M. S., Naldini, L., Honjo, T., Kokaia, Z., Shupliakov, O., Cassidy, R. M., Lindvall, O., and Frisen, J. (2009). Forebrain ependymal cells are Notch-dependent and generate neuroblasts and astrocytes after stroke. Nat. Neurosci. 12, 259-267.
Caruso, M., Panis, Y., Gagandeep, S., Houssin, D., Salzmann, J. L., and Klatzmann, D. (1993). Regression of established macroscopic liver metastases after in situ transduction of a suicide gene. Proc. Natl. Acad. Sci. U. S. A 90, 7024-7028.
Chen, Y. W., Chenier, I., Chang, S. Y., Tran, S., Ingelfinger, J. R., and Zhang, S. L. (2011). High glucose promotes nascent nephron apoptosis via NF-kappaB and p53 pathways. Am. J. Physiol Renal Physiol 300, F147-F156.
Cheng, P., Zlobin, A., Volgina, V., Gottipati, S., Osborne, B., Simel, E. J., Miele, L., and Gabrilovich, D. I. (2001). Notch-1 regulates NF-kappaB activity in hemopoietic progenitor cells. J. Immunol. 167, 4458-4467.
Coll, A. P., Farooqi, I. S., and O'Rahilly, S. (2007). The hormonal control of food intake. Cell 129, 251-262.
Culver, K. W., Ram, Z., Wallbridge, S., Ishii, H., Oldfield, E. H., and Blaese, R. M. (1992). In vivo gene transfer with retroviral vector-producer cells for treatment of experimental brain tumors. Science 256, 1550-1552.
Dutta, J., Fan, Y., Gupta, N., Fan, G., and Gelinas, C. (2006). Current insights into the regulation of programmed cell death by NF-kappaB. Oncogene 25, 6800-6816.
Emsley, J. G., Mitchell, B. D., Kempermann, G., and Macklis, J. D. (2005). Adult neurogenesis and repair of the adult CNS with neural progenitors, precursors, and stem cells. Prog. Neurobiol. 75, 321-341.
Espinosa, L., Ingles-Esteve, J., Robert-Moreno, A., and Bigas, A. (2003). IkappaBalpha and p65 regulate the cytoplasmic shuttling of nuclear corepressors: cross-talk between Notch and NFkappaB pathways. Mol. Biol. Cell 14, 491-502.
Flier, J. S. (2006). Neuroscience. Regulating energy balance: the substrate strikes back. Science 312, 861-864.
Gage, F. H. (2000). Mammalian neural stem cells. Science 287, 1433-1438.
Garcia, A. D., Doan, N. B., Imura, T., Bush, T. G., and Sofroniew, M. V. (2004). GFAP-expressing progenitors are the principal source of constitutive neurogenesis in adult mouse forebrain. Nat. Neurosci. 7, 1233-1241.
Gould, E. (2007). How widespread is adult neurogenesis in mammals? Nat. Rev. Neurosci. 8, 481-488.
Gross, C. G. (2000). Neurogenesis in the adult brain: death of a dogma. Nat. Rev. Neurosci. 1, 67-73.
Hayden, M. S., West, A. P., and Ghosh, S. (2006). SnapShot: NF-kappaB signaling pathways. Cell 127, 1286-1287.
Hoffmann, A. and Baltimore, D. (2006). Circuitry of nuclear factor kappaB signaling. Immunol. Rev. 210, 171-186.
Hotamisligil, G. S. (2006). Inflammation and metabolic disorders. Nature 444, 860-867.
Huang, W. C., Chen, J. J., and Chen, C. C. (2003). c-Src-dependent tyrosine phosphorylation of IKKbeta is involved in tumor necrosis factor-alpha-induced intercellular adhesion molecule-1 expression. J. Biol. Chem. 278, 9944-9952.
is-Donini, S., Dellarole, A., Crociara, P., Francese, M. T., Bortolotto, V., Quadrato, G., Canonico, P. L., Orsetti, M., Ghi, P., Memo, M., Bonini, S. A., Ferrari-Toninelli, G., and Grilli, M. (2008). Impaired adult neurogenesis associated with short-term memory defects in NF-kappaB p50-deficient mice. J. Neurosci. 28, 3911-3919.
Johansson, C. B., Momma, S., Clarke, D. L., Risling, M., Lendahl, U., and Frisen, J. (1999). Identification of a neural stem cell in the adult mammalian central nervous system. Cell 96, 25-34.
Karin, M. and Lin, A. (2002). NF-kappaB at the crossroads of life and death. Nat. Immunol. 3, 221-227.
Kleinridders, A., Schenten, D., Konner, A. C., Belgardt, B. F., Mauer, J., Okamura, T., Wunderlich, F. T., Medzhitov, R., and Bruning, J. C. (2009). MyD88 signaling in the CNS is required for development of fatty acid-induced leptin resistance and diet-induced obesity. Cell Metab 10, 249-259.
Koch, P., Kokaia, Z., Lindvall, O., and Brustle, O. (2009). Emerging concepts in neural stem cell research: autologous repair and cell-based disease modelling. Lancet Neurol. 8, 819-829.
Kokoeva, M. V., Yin, H., and Flier, J. S. (2005). Neurogenesis in the hypothalamus of adult mice: potential role in energy balance. Science 310, 679-683.
Kokoeva, M. V., Yin, H., and Flier, J. S. (2007). Evidence for constitutive neural cell proliferation in the adult murine hypothalamus. J. Comp Neurol. 505, 209-220.
Koo, J. W. and Duman, R. S. (2008). IL-1beta is an essential mediator of the antineurogenic and anhedonic effects of stress. Proc. Natl. Acad. Sci. U. S. A 105, 751-756.
Koo, J. W., Russo, S. J., Ferguson, D., Nestler, E. J., and Duman, R. S. (2010). Nuclear factor-kappaB is a critical mediator of stress-impaired neurogenesis and depressive behavior. Proc. Natl. Acad. Sci. U. S. A 107, 2669-2674.
Kuhn, H. G., ckinson-Anson, H., and Gage, F. H. (1996). Neurogenesis in the dentate gyrus of the adult rat: age-related decrease of neuronal progenitor proliferation. J. Neurosci. 16, 2027-2033.
Li, Q. and Verma, I. M. (2002). NF-kappaB regulation in the immune system. Nat. Rev. Immunol. 2, 725-734.
Lie, D. C., Dziewczapolski, G., Willhoite, A. R., Kaspar, B. K., Shults, C. W., and Gage, F. H. (2002). The adult substantia nigra contains progenitor cells with neurogenic potential. J. Neurosci. 22, 6639-6649.
Lin, K. I., DiDonato, J. A., Hoffmann, A., Hardwick, J. M., and Ratan, R. R. (1998). Suppression of steady-state, but not stimulus-induced NF-kappaB activity inhibits alphavirus-induced apoptosis. J. Cell Biol. 141, 1479-1487.
Lindvall, O. and Kokaia, Z. (2006). Stem cells for the treatment of neurological disorders. Nature 441, 1094-1096.
Lois, C. and varez-Buylla, A. (1993). Proliferating subventricular zone cells in the adult mammalian forebrain can differentiate into neurons and glia. Proc. Natl. Acad. Sci. U. S. A 90, 2074-2077.
Louvi, A. and rtavanis-Tsakonas, S. (2006). Notch signalling in vertebrate neural development. Nat. Rev. Neurosci. 7, 93-102.
Lutolf, S., Radtke, F., Aguet, M., Suter, U., and Taylor, V. (2002). Notchl is required for neuronal and glial differentiation in the cerebellum. Development 129, 373-385.
Markakis, E. A., Palmer, T. D., Randolph-Moore, L., Rakic, P., and Gage, F. H. (2004). Novel neuronal phenotypes from neural progenitor cells. J. Neurosci. 24, 2886-2897.
Martino, G. and Pluchino, S. (2006). The therapeutic potential of neural stem cells. Nat. Rev. Neurosci. 7, 395-406.
Meng, Q. and Cai, D. (2011). Defective hypothalamic autophagy directs the central pathogenesis of obesity via IKK-beta/NF-kappaB pathway. J. Biol. Chem.
Morrison, S. J. (2001). Neuronal potential and lineage determination by neural stem cells. Curr. Opin. Cell Biol. 13, 666-672.
Morshead, C. M., Reynolds, B. A., Craig, C. G., McBurney, M. W., Staines, W. A., Morassutti, D., Weiss, S., and van der, K. D. (1994). Neural stem cells in the adult mammalian forebrain: a relatively quiescent subpopulation of subependymal cells. Neuron 13, 1071-1082.
Mu, Y., Lee, S. W., and Gage, F. H. (2010). Signaling in adult neurogenesis. Curr. Opin. Neurobiol.
Munzberg, H. and Myers, M. G., Jr. (2005). Molecular and anatomical determinants of central leptin resistance. Nat. Neurosci. 8, 566-570.
Niswender, K. D., Baskin, D. G., and Schwartz, M. W. (2004). Insulin and its evolving partnership with leptin in the hypothalamic control of energy homeostasis. Trends Endocrinol. Metab 15, 362-369.
Oakley, F., Mann, J., Ruddell, R. G., Pickford, J., Weinmaster, G., and Mann, D. A. (2003). Basal expression of IkappaBalpha is controlled by the mammalian transcriptional repressor RBP-J (CBF1) and its activator Notchl. J. Biol. Chem. 278, 24359-24370.
Okada, Y., Shimazaki, T., Sobue, G., and Okano, H. (2004). Retinoic-acid-concentration-dependent acquisition of neural cell identity during in vitro differentiation of mouse embryonic stem cells. Dev. Biol. 275, 124-142.
Oya, S., Yoshikawa, G., Takai, K., Tanaka, J. I., Higashiyama, S., Saito, N., Kirino, T., and Kawahara, N. (2009). Attenuation of Notch signaling promotes the differentiation of neural progenitors into neurons in the hippocampal CAl region after ischemic injury. Neuroscience 158, 683-692.
Palmer, T. D., Markakis, E. A., Willhoite, A. R., Safar, F., and Gage, F. H. (1999). Fibroblast growth factor-2 activates a latent neurogenic program in neural stem cells from diverse regions of the adult CNS. J. Neurosci. 19, 8487-8497.
Palmer, T. D., Ray, J., and Gage, F. H. (1995). FGF-2-responsive neuronal progenitors reside in proliferative and quiescent regions of the adult rodent brain. Mol. Cell Neurosci. 6, 474-486.
Pencea, V., Bingaman, K. D., Wiegand, S. J., and Luskin, M. B. (2001). Infusion of brain-derived neurotrophic factor into the lateral ventricle of the adult rat leads to new neurons in the parenchyma of the striatum, septum, thalamus, and hypothalamus. J. Neurosci. 21, 6706-6717.
Pierce, A. A. and Xu, A. W. (2010). De novo neurogenesis in adult hypothalamus as a compensatory mechanism to regulate energy balance. J. Neurosci. 30, 723-730.
Pluchino, S., Zanotti, L., Rossi, B., Brambilla, E., Ottoboni, L., Salani, G., Martinello, M., Cattalini, A., Bergami, A., Furlan, R., Comi, G., Constantin, G., and Martino, G. (2005). Neurosphere-derived multipotent precursors promote neuroprotection by an immunomodulatory mechanism. Nature 436, 266-271.
Posey, K. A., Clegg, D. J., Printz, R. L., Byun, J., Morton, G. J., Vivekanandan-Giri, A., Pennathur, S., Baskin, D. G., Heinecke, J. W., Woods, S. C., Schwartz, M. W., and Niswender, K. D. (2009). Hypothalamic proinflammatory lipid accumulation, inflammation, and insulin resistance in rats fed a high-fat diet. Am. J. Physiol Endocrinol. Metab 296, E1003-E1012.
Purkayastha, S., Zhang, G., and Cai, D. (2011a). Uncoupling the mechanisms of obesity and hypertension by targeting hypothalamic IKK-beta and NF-kappaB. Nat. Med. 17, 883-887.
Purkayastha, S., Zhang, H., Zhang, G., Ahmed, Z., Wang, Y., and Cai, D. (2011b). Neural dysregulation of peripheral insulin action and blood pressure by brain endoplasmic reticulum stress. Proc. Natl. Acad. Sci. U. S. A 108, 2939-2944.
Qin, Z. H., Wang, Y., Nakai, M., and Chase, T. N. (1998). Nuclear factor-kappa B contributes to excitotoxin-induced apoptosis in rat striatum. Mol. Pharmacol. 53, 33-42.
Ray, J., Peterson, D. A., Schinstine, M., and Gage, F. H. (1993). Proliferation, differentiation, and long-term culture of primary hippocampal neurons. Proc. Natl. Acad. Sci. U. S. A 90, 3602-3606.
Reynolds, B. A. and Weiss, S. (1992). Generation of neurons and astrocytes from isolated cells of the adult mammalian central nervous system. Science 255, 1707-1710.
Rolls, A., Shechter, R., London, A., Ziv, Y., Ronen, A., Levy, R., and Schwartz, M. (2007). Toll-like receptors modulate adult hippocampal neurogenesis. Nat. Cell Biol. 9, 1081-1088.
rtavanis-Tsakonas, S., Rand, M. D., and Lake, R. J. (1999). Notch signaling: cell fate control and signal integration in development. Science 284, 770-776.
Ruan, H. and Lodish, H. F. (2004). Regulation of insulin sensitivity by adipose tissue-derived hormones and inflammatory cytokines. Curr. Opin. Lipidol. 15, 297-302.
Ryan, K. M., Ernst, M. K., Rice, N. R., and Vousden, K. H. (2000). Role of NF-kappaB in p53-mediated programmed cell death. Nature 404, 892-897.
Shoelson, S. E. and Goldfine, A. B. (2009). Getting away from glucose: fanning the flames of obesity-induced inflammation. Nat. Med. 15, 373-374.
Suh, H., Consiglio, A., Ray, J., Sawai, T., D'Amour, K. A., and Gage, F. H. (2007). In vivo fate analysis reveals the multipotent and self-renewal capacities of Sox2+ neural stem cells in the adult hippocampus. Cell Stem Cell 1, 515-528.
Takahashi, K. and Yamanaka, S. (2006). Induction of pluripotent stem cells from mouse embryonic and adult fibroblast cultures by defined factors. Cell 126, 663-676.
Temple, S. (2001). The development of neural stem cells. Nature 414, 112-117.
Tiberghien, P. (1998). “Suicide” gene for the control of graft-versus-host disease. Curr. Opin. Hematol. 5, 478-482.
Vacca, A., Felli, M. P., Palermo, R., Di, M. G., Calce, A., Di, G. M., Frati, L., Gulino, A., and Screpanti, I. (2006). Notch3 and pre-TCR interaction unveils distinct NF-kappaB pathways in T-cell development and leukemia. EMBO J. 25, 1000-1008.
Vallieres, L., Campbell, I. L., Gage, F. H., and Sawchenko, P. E. (2002). Reduced hippocampal neurogenesis in adult transgenic mice with chronic astrocytic production of interleukin-6. J. Neurosci. 22, 486-492.
varez-Buylla, A. and Lim, D. A. (2004). For the long run: maintaining germinal niches in the adult brain. Neuron 41, 683-686.
Vousden, K. H. (2009). Partners in death: a role for p73 and NF-kB in promoting apoptosis. Aging (Albany. N.Y.) 1, 275-277.
Wernig, M. and Brustle, O. (2002). Fifty ways to make a neuron: shifts in stem cell hierarchy and their implications for neuropathology and CNS repair. J. Neuropathol. Exp. Neurol. 61, 101-110.
Whitman, M. C. and Greer, C. A. (2009). Adult neurogenesis and the olfactory system. Prog. Neurobiol. 89, 162-175.
Yang, X., Klein, R., Tian, X., Cheng, H. T., Kopan, R., and Shen, J. (2004). Notch activation induces apoptosis in neural progenitor cells through a p53-dependent pathway. Dev. Biol. 269, 81-94.
Yuan, M., Konstantopoulos, N., Lee, J., Hansen, L., Li, Z. W., Karin, M., and Shoelson, S. E. (2001). Reversal of obesity- and diet-induced insulin resistance with salicylates or targeted disruption of Ikkbeta. Science 293, 1673-1677.
Zhang, G., Bai, H., Zhang, H., Dean, C., Wu, Q., Li, J., Guariglia, S., Meng, Q., and Cai, D. (2011). Neuropeptide exocytosis involving synaptotagmin-4 and oxytocin in hypothalamic programming of body weight and energy balance. Neuron 69, 523-535.
Zhang, X., Zhang, G., Zhang, H., Karin, M., Bai, H., and Cai, D. (2008). Hypothalamic IKKbeta/NF-kappaB and ER stress link overnutrition to energy imbalance and obesity. Cell 135, 61-73.

1-19. (canceled)

20. A method of enhancing the survival of hypothalamic stem cells in a mammalian subject having obesity or an obesity comorbidity comprising administering centrally to the mammalian subject an inducible pluripotent cell having a IKKβ genetic sequence deleted therein or a neural stem cell having a IKKβ genetic sequence deleted therein effective to enhance survival of hypothalamic stem cells in a mammalian subject.

21. The method of claim 20, wherein the inducible pluripotent cell is administered.

22. The method of claim 20, wherein the neural stem cell is administered.

23. The method of claim 21, wherein the inducible pluripotent cell is a human or a human-derived cell.

24. The method of claim 22, wherein the neural stem cell is a human or a human-derived cell.

25. The method of claim 22, wherein the neural stem cell is a hypothalamic stem cell.

26. The method of claim 22, wherein the obesity comorbidity is type 2 diabetes.
